(19) United States
(12) Patent Application Publication
(10) Pub. No.: US 2010/0086482 A1
(43) Pub. Date: Apr. 8, 2010

(54) DIVERGENT SYNTHESIS OF LOOPED POLY(ESTER)-AND POLY(ETHER)-SUBSTITUTED DENDRONS AND DENDRIMERS

(75) Inventors: Donald A. Tomalia, Midland, MI (US); Douglas R. Swanson, Mt. Pleasant, MI (US); Baohua Huang, Ann Arbor, MI (US); Veera Reddy Pulgam, Mt. Pleasant, MI (US)

Correspondence Address: TECHNOLOGY LAW, PLLC, 3595 N. SUNSET WAY, SANFORD, MI, 48657, US

(73) Assignee: Dendritic Nanotechnologies, Inc., Mt. Pleasant, MI (US)

(21) Appl. No.: 12/448,513

(22) PCT Filed: Dec. 28, 2007
(86) PCT No.: PCT/US07/26445

§ 371 Date: Jun. 23, 2009

Related U.S. Application Data
(60) Provisional application No. 60/878,025, filed on Dec. 29, 2006.

(51) Int. Cl. A61K 49/00; C07D 487/00; C07D 491/00; A61K 31/395; C07D 245/00; A61K 31/495; A61K 31/16; G01N 33/566

(52) U.S. Cl. 424/9.1; 540/469; 514/450; 540/467; 540/460; 564/135; 514/250; 514/616; 436/94

(57) ABSTRACT

The present invention describes a process for preparing new looped dendrimer and dendron compounds by controlling the molar amount of branch cell reagent monomer that is combined with various cores bearing core-XR functionalities (e.g., primary, or secondary amines, thiol, or epoxy functionalities). These looped, macrocyclic structures are more robust to various conditions, with greater resistance to acid/base hydrolysis. Alternatively, the looped, macrocyclic structure may offer new orientations that would qualify it as a better chelation ligand for metals, and other similar uses.

### BACKGROUND OF THE INVENTION

1. Field of the Invention

This invention relates to the field of dendritic polymers and their preparation. Specifically this invention concerns the use of divergent synthesis for the preparation of dendrimers.

2. Description of Related Art

Two very similar “divergent strategies” provided the first examples of both small dendritic structures (i.e., Vögtle type, poly(propyleneimine) (PPI) cascade structures) [see Vögtle, et al., Synthesis, 155 (1978)] and macromolecular dendritic structures (i.e., Tomalia type, poly(amidoamine), (PAMAM) dendrimer structures) [see Tomalia, et al., Poly. J., 17, 117 (1985)]. Each of these iteration strategies involved exhaustive addition of linear-α,β-unsaturated monomers (i.e., acrylonitrile or methyl acrylate monomers, respectively), to all active hydrogens on the α,ω-alkylenediamine cores and subsequent amine terminated generations. These Michael addition stages provided the “critical amplification step” in the so-called “in situ” branch cell construction approach. (See FIG. 1.) In fact, exhaustive addition to such active amine hydrogen moieties is essential at each iteration step in order to obtain defect free, ideal dendrimer structures that are defined by perfect symmetry and fulfillment of growth parameters that may be predicted using the well known “dendritic branching mathematics” for dendrimers [e.g., Tomalia, et al., Angew. Chem. Int. Ed. Engl., 138 (1990)].

In the “genealogically directed synthesis” [see, for example, Dvornic, P. R. and Tomalia, D. A., Macromol. Sym., 98, 403-428 (1995); Tomalia, D. A., Adv. Mater. 6, 529 (1994)] of PAMAM dendrimers, only minor generational deviations from ideal structures (i.e., polyvalent stoichiometries) are observed in the early generations (i.e. G=b 0-4). These dendrimer defects have been generally attributed to geometrically controlled, intramolecular looping events to form macrocyclic moieties or sterically influenced or sterically induced stoichiometry (SIS) type events to produce missing arm type products. These looping events produced mass defects of 60 daltons per event, whereas a mass defect of 114 daltons was observed for each (SIS) type event. In each case these two effects not only led to mass defects from theoretical but also gave exponentially decreased dendrimer terminal valency as a function of generation. The suppressed valency and masses exhibited by these geometrically controlled, macrocyclic products has been referred to as “geometrically induced stoichiometry” (GIS). [See Brendia I. Schwartz, et al., Rapid Comm. Mass Spec., 9, 1552-1555 (1995).] Similarly, the “constrained reaction space phenomenon” leading to mass and valency deficient dendrimers has been referred to as “sterically induced stoichiometry” (SIS) [see Dvornic, P. R. and Tomalia, D. A., Macromol. Sym., 98, 403-428 (1995)]. Serious mass and stoichiometry deviations from theoretical values for typical (core [C]; EDA); dendri-PAMAM-(NH₂)_{z} type dendrimers due to GIS effects are not observed until approximately generation G=4-5. In this series, more substantial mass and stoichiometry defects were noted beyond generation G=7; undoubtedly indicating both GIS effects and the substantial onset of de Gennes dense packing or SIS behavior [see Tomalia, et al., Angew. Chem. Int. Ed. Engl., 29, 138 (1990)]. This latter effect is attributed to the more sterically constrained relationship of terminal primary amines with their neighbors.

It is apparent that the stoichiometries (i.e. outer shell valencies) of these higher generation PAMAM dendrimers are systematically decreasing as a function of generation, undoubtedly due to GIS as well as SIS type events. Similar SIS-type products have been observed when protected, monovalent, branch cell monomers (i.e., AB_{z} type, see (a) in FIG. 1) were used for the synthesis of dendri-poly(ethers) [see Tomalia, et al., J. Org. Chem. 52, 5305 (1987)], dendri-(poly thioethers) or dendri-poly(amides) (arborols). In each case the mass defects and surpressed stoichiometries were not observed until iteration was attempted at G=2 to 3.

In the preparation of known PAMAM dendrimers there has been observed space related effects [i.e., “sterically induced stoichiometries” (SIS) controlled products] and geometry controlled products [i.e., “geometrically induced stoichiometries” (GIS)] effects in the production of traditional PAMAM dendrimers. [See, for example, P. R. Dvornic and D. A. Tomalia, Macromol. Symp., 403-428 (1995).] Usually these effects do not occur until G=4 or higher. In each case, digressions from ideal dendrimer masses and terminal functionality are observed as a function of generation.

In the case of GIS effects, appropriate geometries occur between the “branch cell reagent (monomer)” and the reacting substrate that tend to reduce entropy barriers to such a level that looping or macrocyclic structure formation is favored. When this occurs, it produces a dendritic growth/connectivity that exhibits lower mass and terminal functionality values than theory. In some instances this GIS type change in connectivity can be a benefit in that it may produce multiple connectivity linkages between the core and a terminal group which may lead to a more robust structure with greater resistance to acid/base hydrolysis. Alternatively, the looped, macrocyclic structure may offer new orientations that would qualify it as a better chelation ligand for metals, and other similar uses.

In a recent article by K. D. Zhang, et al., J. Applied Polym. Sci., 92, 1018-1022, (2004) there is reported a single example which describes the production of an ideal, G=1, acrylate terminated tetra-dendron poly(ester-acrylate) dendrimer when reacting trimethylolpropane triglycidyl ether (TMPTA) with ethylenediamine. No mention is made of SIS controlled tri-dendron or GIS controlled, looped dendrimers.

A recent article by Dong-mei Xu, et al., Ganguang Kexue Yu Guang Huaxue, 22(4), 287-292 (2004) discusses, in its English abstract, the formation of quick UV-curing dendritic acrylate oligomers having multiple acrylate double bonds prepared from ethylenediamine and TMPTA using methanol as a catalyst. No mention is made of SIS controlled tri-dendron or GIS controlled, looped dendrimers.

In a related article by Dong-mei Xu, et al., J. Appl. Polymer Sci., 92(2), 1018-1022 (2004) they discuss a similar process for curing speed of a related dendritic acrylate oligomer with eight double bonds to the ones they made before. No mention is made of SIS controlled tri-dendron or GIS controlled, looped dendrimers.

In an English abstract by Chun-hua Ning, et al., Jingxi Huagong, 19(11), 631-633, 643 (2002), they discuss using divergent synthesis for dendrimers using ethylenediamine and TMPTA to obtain a higher UV curing rate for the polymer. No mention is made of SIS controlled tri-dendron or GIS controlled, looped dendrimers.

Clearly, to have a PAMAM or PEHAM dendrimer made which is robust and displays better chelation and/or encapsulation properties than previously known dendrimers as discussed above would be desirable.

### BRIEF SUMMARY OF THE INVENTION

This invention describes the use of polyvalent, nanoscale monomers in a “non-protect” divergent strategy. These syntheses of (amine:core);G=1;[dendri-polyester/acrylate/amine)] and (epoxy:core); G=1; [dendri-poly(etherhydroxylamine)] type dendrimers allow many advantages over the traditional divergent methodologies. Foremost among the advantages are commercial availability of branched acrylate or amine monomers and the ease of dendrimer isolation and scalability. SIS-type, GIS-type and ideal dendrimer structures are possible by merely controlling core design and unprotected (Aₓ):core —NH functionality ratios in the core dendronization (via Michael addition or epoxy ring opening) step leading to G=1; dendri-poly(ester/acrylates) or dendri-poly(etherhydroxylamines). These transformations clearly illustrate the importance of previously described sterically induced stoichiometry, SIS-type products [e.g., WO2006/115547; WO2006/065266] and geometrically controlled events leading to geometrically induced stoichiometry GIS-type products as well as ideal dendrimer structures. Quite remarkably, the nanoscale nature of the (Aₓ) type reagents used in this strategy appear to dramatically induce SIS and GIS events much earlier than has ever been observed in traditional divergent, iteration schemes using smaller, sub-nanoscale reagents.

The present invention particularly concerns dendrimers and dendrons comprising a looped, macrocyclic structure in one or more branches of the formula

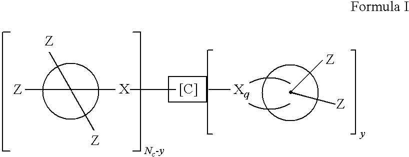

wherein:
   [C] is the core of the dendrimer or dendron that may have more than 1 branch arm depending on the number of core-XR functionalities present;
   XR represents the core functionalities and when R═H is a mono-alkyl amine, an α,ω-alkylenediamine, a poly(thiol) moiety, a mono- or poly-alkylene-X moiety, or when R═C is a strained ring having at least 1× moiety as part of the ring;
   X is N, S or O;
   N_{c} is the multiplicity of the core and is from 1 to 1,000;
   y is the number of macrocyclic loops and is from 1 to Z/2;
   q is from 1-3;
   Z is the surface of the dendrimer or dendron and is a poly(ester)- or poly(ether)-substituted terminal functionality, which may be from 1 to the theoretical number possible.

The dendrimers or dendrons of Formula I wherein Z is poly(ester)- or poly(ether)- that is substituted with an acrylate, amine, piperazine, epoxy, aziridine, thioether or morpholine moiety or any functionality derived from nucleophilic addition or reaction with an epoxy, acrylate or aziridine moiety are preferred.

In Formula (I) some preferred embodiments are those where: y is 1 or 2; or the core is a mono-alkyl amine, an α,ω-alkylenediamine, a polyalkylene amine, a poly(thiol), or a poly(epoxy) core; or X is N or an epoxy ring.

The process for making the dendrimers and dendrons of Formula I are by reacting a dendrimer or dendron core having one or more core-XR functionalities (where X═N (primary or secondary) or S or O and R═H) or a strained ring (where X═N, S, or O as a part of a constrained ring of carbon atoms and R═C of the strained ring) available for reaction with a branch cell reagent, where the molar ratio of branch cell reagent to core-XR functionality is from about 0.5:1 to about 2:1, in the presence of a suitable solvent.

In Formula (I) some embodiments for the dendrimers and dendrons and the process to make them are those where the core is EDA, HDA, DETA, TREN, PETGE, TMPTGE, PEHAM, PAMAM or PEI; Z=surface group terminal functionality and is a poly(ester)- or poly(ether)-(acrylate, amine, piperazine, epoxy, aziridine, thioether, or morpholine) moiety; the branch cell reagent is PTA, PETGE, TMPTA, TMPTGE, PPT, TREN or DETA; and/or the solvent is methanol. A further embodiment of Formula (I) is where the core is a mono-alkyl amine core and the branch cell reagent is PTA, PETGE, TMPTA, PPT or TMPTGE and the molar ratio of branch cell reagent to core-XR functionality is from about 0.5:1 to about 1:1. Another embodiment of Formula (I) is where the core is an α,ω-alkylenediamine core, the branch cell reagent is PTA, PETGE, TMPTA, PPT or TMPTGE and the molar ratio of branch cell reagent to core-XR functionality is about 2:1.

### BRIEF DESCRIPTION OF THE DRAWINGS

FIG. 1 illustrates the Michael addition stages that provide the “critical amplification step” in the so-called “in situ” branch cell construction approach.

FIG. 2 illustrates the use of a hexyldiamine core, which is a larger core, where all core NH can react as there is sufficient space.

FIG. 3 illustrates the use of a smaller core, EDA, where only 3 NH react. A smaller reagent can then react with the remaining NH.

FIG. 4 illustrates the EDA core with a smaller reagent (i.e., methyl acrylate) to have all 4 NH groups reacted.

FIG. 5 shows NH₄OH+MeOH (1:1) (ninhydrin spray) of the reaction of Example 25; Synthesis of G=1.0 epoxy dendrimer [15] from PETGE [C] and PETGE-PIPZ [14].

FIG. 6 shows the PAGE of Example 28: 10% Homogeneous Gel where Lane 1, [24] (G=1.0), Lane 2, (G=2.0, TMPTGE [D] core), Lane 3, (G=2.0, PETGE [C] core).

### DETAILED DESCRIPTION OF THE INVENTION

### Glossary

The following terms as used in this application are to be defined as stated below and for these terms, the singular includes the plural.
Alkyl means any number of carbon atoms for the term that is used, whether linear or branched, alone or part of another term such as alkyl substituted, alkylaryl, cycloalkyl, heterocyclic moieties, and others; typically from C₁-C₁₀₀, with C₁-C₅₀ preferred and C₁-C₂₅ most preferred. In a similar manner alkene and alkyne are defined broadly; typically from C₂-C₂₀₀, with C₂-C₁₀₀ preferred.
AmA means amylamine, 99% by Aldrich
amu means atomic mass units
C means the core of the dendrimer polymer
DADD means 1,12-diaminododecane, 97% by Fluka
DCM means dichloromethane
Dendrimer means any dendritic structure which contains a core with a multiplicity of 2 or greater (i.e., N_{c}≧2) and will bear 2 or more dendrons
Dendron means any dendritic structure where the core multiplicity is one (i.e., N_{c}=1)
DETA means diethylenetriamine
DI means deionized water (18.2 MΩ)
DME means ethyleneglycol dimethyl ether
DMSO means dimethylsulfoxide, Acros organics
EDA means ethylenediamine, 99.5% by Aldrich
equiv. means equivalent(s)
FT-IR means Fourier Transform Infrared Spectroscopy
G means dendrimer generation, which is indicated by the number of concentric branch cell walls surrounding the core (C), usually counted sequentially from the core (C)
g means gram(s)
GIS means geometrically induced stoichiometry
HDA means 1,6-hexanediamine, 99.5% by Aldrich
HPLC means high pressure liquid chromatography
L means liter(s)
MALDI-TOF means matrix-assisted laser desorption ionization time of flight mass spectroscopy
MeOH means methanol; 99.8% by Aldrich
mg means milligram(s)
mins. means minutes
mL means milliter(s)
N_{b} means branch cell multiplicity or branch cell multiplicity
N_{c} means core multiplicity or core valency
NMR means nuclear magnetic resonance
PAGE means poly(acrylamide) gel electrophoresis
PAMAM means poly(amidoamine), including linear and branched polymers or dendrimers with primary amine terminal groups
PEA means methyl isobutyl protected 1-(2-aminoethyl)piperazine; or poly(ester-acrylate) polymers or dendrimers
PEHAM means poly(etherhydroxylamine) polymers or dendrimers
PEI means poly(ethyleneimine)
PPT means propargyl pentaerythritol triglycidyl ether
PTA means pentaerythritol tetraacrylate, Aldrich
PETGE means pentaerythritol tetraglycidyl ether
PIPZ means piperazine or diethylenediamine
Rf means relative flow in TLC
RT means ambient temperature or room temperature, about 20-25° C.
SEC means size exclusion chromatography
SIS means sterically induced stoichiometry
Strained ring means a C3 or C4 ring where at least 1 N, S or O is a part of the ring
TLC means thin layer chromatography
TMPTA means trimethylolpropane triacrylate
TMPTGE means trimethylolpropane triglycidyl ether; Aldrich
TREN means tris-(2-aminoethyl) amine
UF means ultraviolet and visible spectroscopy
Z means dendron or dendrimer terminal functionality groups where the theoretical maximum number of groups possible is determined by the generation G and the multiplicity of the branching reagent

### Discussion

The present invention describes the addition of branched (A)ₓ monomers (i.e., TMPTA; x=3 acrylates or PTA; x=4 acrylates or TREN; x=4 primary amines) to various mono- and poly-alkyleneamine or poly-epoxide cores under mild conditions to produce; (core: amine); G=1; [dendri-poly(ester-acrylate)_{z}] (PEA) or (core: epoxy); G=1; [dendri-poly(etherhydroxylamine)_{z}] (PEHAM) type dendrimers in high yields. Quite remarkably, this strategy did not necessitate “large excess reagent protocols” as required for traditional, known PAMAM dendrimer syntheses, yet still produced relatively little oligomeric/polymeric side product. However, “large excess reagent protocols” did favor products exhibiting theoretical (ideal) stoichiometries at the expense of geometrically controlled, macrocyclic (looped) structures when adequate reactivity space was available.

When active amine hydrogens become highly congested on the polyamine core or branching unit, one observes the formation of sterically controlled products. Both the geometrically controlled, GIS, macrocyclic and sterically controlled, SIS, (missing dendron) products exhibited lower terminal stoichiometries and molecular masses compared to ideally dendronized products. It was important to protect the highly reactive acrylate terminated, Michael adduct dendrimers (i.e., (core:amine); G=1; [dendri-poly(ester-acrylates)_{z}]) from light. These reactive intermediates were immediately pacified by reaction of the acrylate functionality with appropriate nucleophilic reagents (i.e., amines or thiols) to avoid polymerization. This procedure thereby produced a shelf stable product that did not polymerize. The dendrimers produced by strained ring-opening reactions (i.e., (core: epoxy); G=1; [dendri-poly(etherhydroxylamine)_{z}] were stable to light and temperature without additional protective steps.

These conversions produced a variety of new terminal functionalized dendrimers. For example, Michael addition of the branched (A)ₓ acrylate monomers onto α,ω-alkylenediamine cores theoretically possessing four dendron attachment sites yielded an ideal tetra-dendron dendrimer only when adequate reaction space was available in concert with adequate branched monomer excess. Surprisingly, it was observed that either unique SIS or GIS effects occurred as one systematically enhanced methylene spacers between the terminal amine functions. This was evidenced by the production of di-, tri- or tetra-dendron containing dendrimers as a function of the core chain length and branched monomer reaction ratios. While not wishing to be bound by theory, it is postulated that introduction of nanoscale (i.e., 1-1.5 nm) branched (A)ₓ monomer reagents onto the sub-nano sized α,ω-alkylenediamine scaffoldings produces a nano-environment with unique “steric and geometric space constraints”. These constraints modulate expected ideal polyvalent modes of amine core reactivity to produce the observed SIS and GIS effects. However, these SIS and GIS effects have never been observed at the core to G=1 conversion stage in dendrimers such as PAMAM and PPI dendrimers.

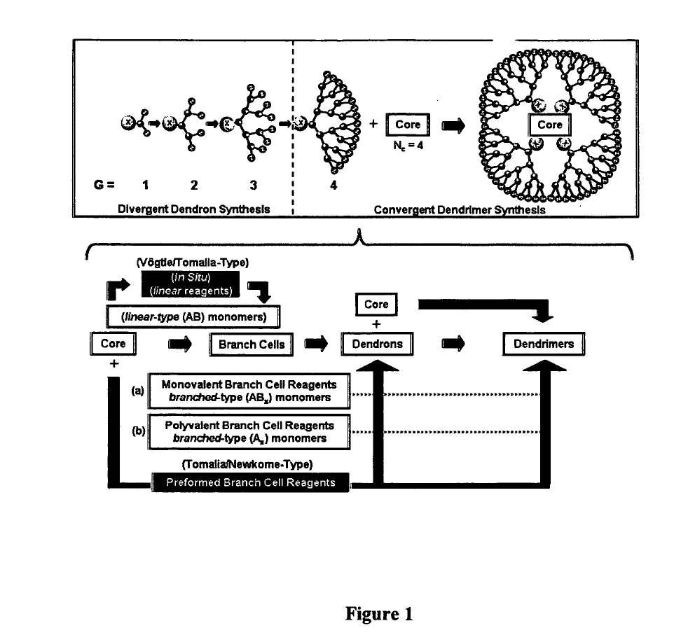

In FIG. 1, route (b) is one of the first examples of a non-protect, (A)ₓ type, branch cell monomer in a divergent dendrimer synthesis. The surprising inducement of major SIS and GIS effects was observed at the very first step (i.e., core to G=1 transition level), while synthesizing a new family of poly(ester-acrylate/amine) (PEA) dendrimers. The first step in this iteration strategy involved dendronization of various mono-alkylamines, linear α,ω-alkylenediamine or poly(alkyleneamine) cores with nanosized, branched poly-acrylate monomers such as TMPTA or PTA. Dendronization involved Michael addition of the respective amine bearing cores to one or more acrylate moieties on the polyvalent branch cell reagent. Reaction of mono-alkylamine cores produced either GIS-type looped, macrocyclic products, ideal dendronized cores, or mixtures of these two architectures, depending on the ratio of core, active amine hydrogen to branched polyacrylate reagent. Linear α,ω-alkylene diamine cores, beginning with EDA, were systematically expanded with methylene spacer groups up to 1,12-diamino-dodecane. Michael addition of the branched tri- or tetra-acrylate monomers, produced spacer directed, tri-dendron functionalized SIS-type dendrimers when the methylene spacer was congested as in EDA. As the methylene groups were enhanced, (i.e. α,ω-butane, hexane or dodecane diamines), either di-dendron functionalized GIS-type, looped (macrocyclic) dendrimers or the expected ideal, tetra-dendron functionalized dendrimers were obtained in very high yields, depending on the molar ratio of branched acrylate monomer/active amine hydrogen. A control reaction with sub-nanosized, linear-methyl acrylate monomer produced the non-SIS, exhaustively alkylated, tetra-substituted Michael adduct in quantitative yield.

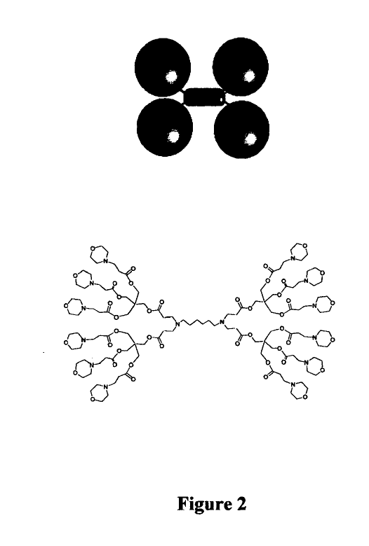

FIG. 2 illustrates the present process for divergent synthesis of ideal dendron structures (where no reactive —NH is present on the core) when hexyldiamine cores, which are a larger core, react with excess branch reagent such as PTA (about 4:1 of branch cell reagent to core-NH). The looped, mono-dendron structure can result when a lower ratio of the branch cell reagent is used.

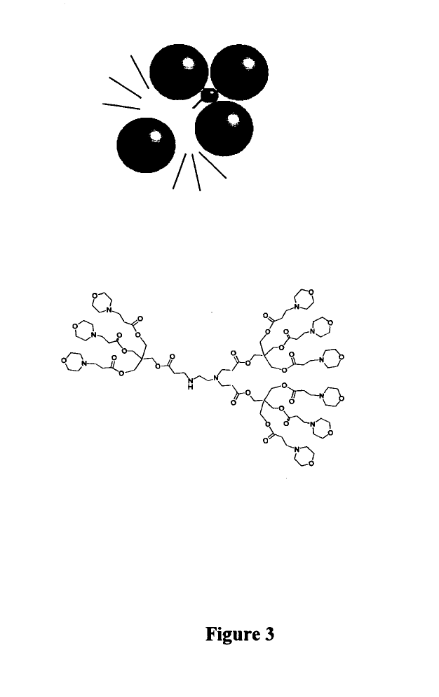

FIG. 3 illustrates the use of a smaller core, EDA, where only three NH react with excess branch reagent, such as PTA, and one reactive —NH remains present in the core.

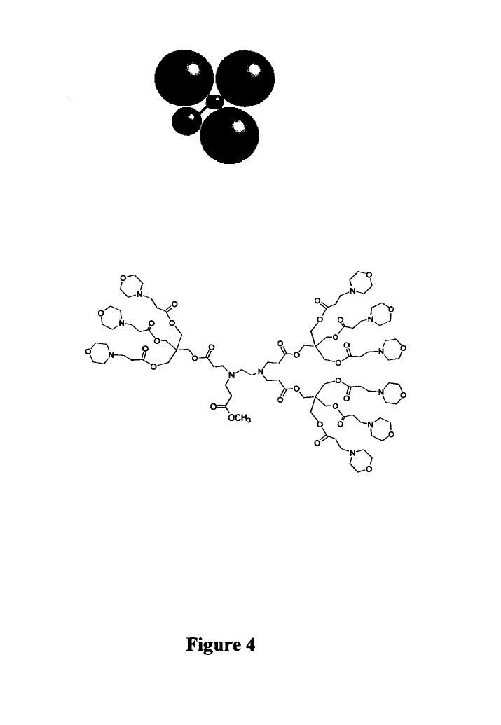

FIG. 4 illustrates the reacted EDA core after additional reaction with a smaller reagent (e.g., methyl acrylate) to have all four NH groups reacted.

The above reactions, disclosed for linear α,ω-diamines, also hold true for linear mono-alkylamine cores and branched poly(alkyleneamine) cores. In addition, these reactions also hold true for other functional groups than acrylate and amine, for example reactions between epoxy and amine.

The dendrons disclosed above have at least one unreacted group on the core (core-XR) or are ideal in structure and have all core functionalities reacted. When the ratio of branch cell reagent to core-functionality is controlled, the dendrons/dendrimers can have looped structures. The unreacted core moiety (core-XR) means a primary amine, secondary amine, thiol moiety, or strained carbon ring structures containing N, S, or O. This lower ratio can vary but may be about 0.5-1:1 of branch cell reagent to core-NH functionality. In the present process for divergent synthesis of tri-dendrons (where one unreacted —NH is present on the core), di-looped, di-dendrons and ideal tetra-dendrons (where no reactive —NH is present on the core) can occur when α,ω-alkylenediamine cores react with branch cell reagents. The di-looped, di-dendrons are formed when the ratio of branch cell reagent to core-NH functionality is about 2:1. In the present process for divergent synthesis of tetra-dendrons (where one unreacted —NH is present on the core), and penta-dendrons (where one reactive —NH is present on the core) when polyalkylene amine cores react with branch cell reagents, no looped dendrons are formed.

### Dendronization of any Polymer Architecture Containing Primary and Secondary Amines

An extension of this present teaching for dendronizing (a) mono-alkylamines, (b) α,ω-diamines and (c) poly(alkyleneamines) would include the dendronization of various polymeric amines. These substrates would include linear PEI, branched polymer architecture possessing reactive/accessible primary or secondary amines (e.g., amine containing linear, branched, dendritic random branched, dendrigraft, dendrons or dendrimer type polymers).

While not wishing to be bound by theory, it is believed that lower excesses of nanoscale sized, branch cell reagents (i.e., acrylate, epoxy or amine monomers) should be added to primary amine or epoxy bearing cores or scaffolding to induce or favor the formation of more looped (i.e., macrocyclic type) products to produce new dendrimer compositions possessing these GIS effect type connectivities/architectures at the core to G=1 conversion stage of PEHAM or PEA type dendrimers. Such looping moieties in the interior of the resulting dendrimer should be expected to give dendrimer compositions which are more resistant to hydrolysis since their connectivity to the core has been enhanced. In the case of branched poly(epoxy) or poly(acrylate) type reagents (monomers) one should expect to produce macrocyclic moieties in the dendrimer interior that may exhibit better chelation and/or encapsulation properties with metals and other appropriate guest molecules.

Dendrimers or dendrons of Formula (I) may be used for a variety of applications. Some of these uses are: as sensors or detection research reagents or for use as in vivo, in vitro or ex vivo diagnostic materials; as transfection agents in research or for use as therapeutic or diagnostic agents; as additives in cosmetic, ink, toner, plastic, paper, or coating applications; as filtration material in water remediation applications; as agents in therapeutic applications; or as chelating agents.

The dendrimers or dendrons of Formula (I) may be form into a chelate or complex that is formed at any location on the dendron or dendrimer (i.e., Core, X, or Z on the dendrimer or dendron) with: (a) metals or their ions (such as transitions metals, radioactive metals, heavy metals, etc.), (b) nucleic acids (such as DNA, siRNA, shRNA, etc.), (c) dyes or pigments (such as organic or inorganic chromophores that absorb or emit radiation in the UV, VIS, IR, microwave, radio frequencies, etc.), (d) pharmaceuticals (such as any active drug or prodrug, etc.), (e) cosmetic ingredients (such as fragrances, vitamins, antioxidants, UV absorbers, etc.) or (f) combinations of these chelates.

For the following examples the various equipment and methods were used to run the various described tests for the results reported in the examples described below.

### Equipment and Methods

### General

Nanoscale, tri- or tetra-branched reagents (i.e., TMPTA [1], PTA [2] or TREN [A]; diameters=1-1.5 nm by CPK models) were utilized as receptor reagents and combined with three different categories of nucleophilic cores, which included: (a) mono-alkyl amines, (b) linear-α,ω-alkylenediamine or (c) branched poly(alkyleneamine) or poly(epoxy) cores. In all cases, high yields of dendrimeric addition products, containing various dendron/macrocyclic looping levels, were obtained under very mild conditions. This divergent process did not require the usual large excesses of branched monomer reagent to avoid oligomer/polymer formation. [See, for example, Dendrimers and other Dendritic Polymers, eds. J. M. J. Fréchet and D. A. Tomalia pub. John Wiley and Sons, 20-23 (2001).]

However, the molar ratio of branched monomer/core functionality could generally be used to control dendronized product architectures. These architectures include: (a) partially looped, GIS-type products; (b) missing dendron, SIS-type products; or (c) ideally dendronized architectures, based on original core stoichiometry. These observations were made within a range of 1-4 molar excess of branched monomer/active core functionality. In some instances, sterically induced stoichiometric SIS-type dendrimer products were obtained, possessing active amine hydrogens that were sterically accessible to small (sub-nano sized) reagents but inaccessible to the nano scale branched acrylate reagents. These products were generally isolated by immiscible solvent extraction or column chromatography. They were characterized by size exclusion chromatographic (i.e., Sephadex™ column) workup, ¹H, ¹³C-NMR, HPLC, MALDI-TOF mass spectrometry and certain diagnostic reagents/reactions to provide further confirmation of structure.

### Branched Acrylate Monomer Reagents

The following structures are the preferred reagents used in the following examples. However, other suitable acrylate monomer reagents can be used.

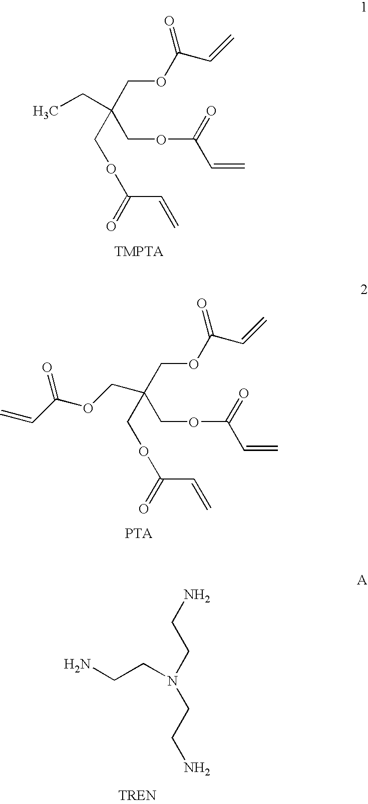

### Mono-Alkylamine Cores

Both short (C₅) and longer (C₁₁) alkyl-substituted mono amine cores were added to either stoichiometric amounts or slight excesses (i.e., up to 4 (Aₓ) moles:core-NH) of the branched tri- or tetra-acrylate monomers in methanol. A variety of dendronized products, depending on monomer excesses, were obtained as shown in Scheme 1 below.

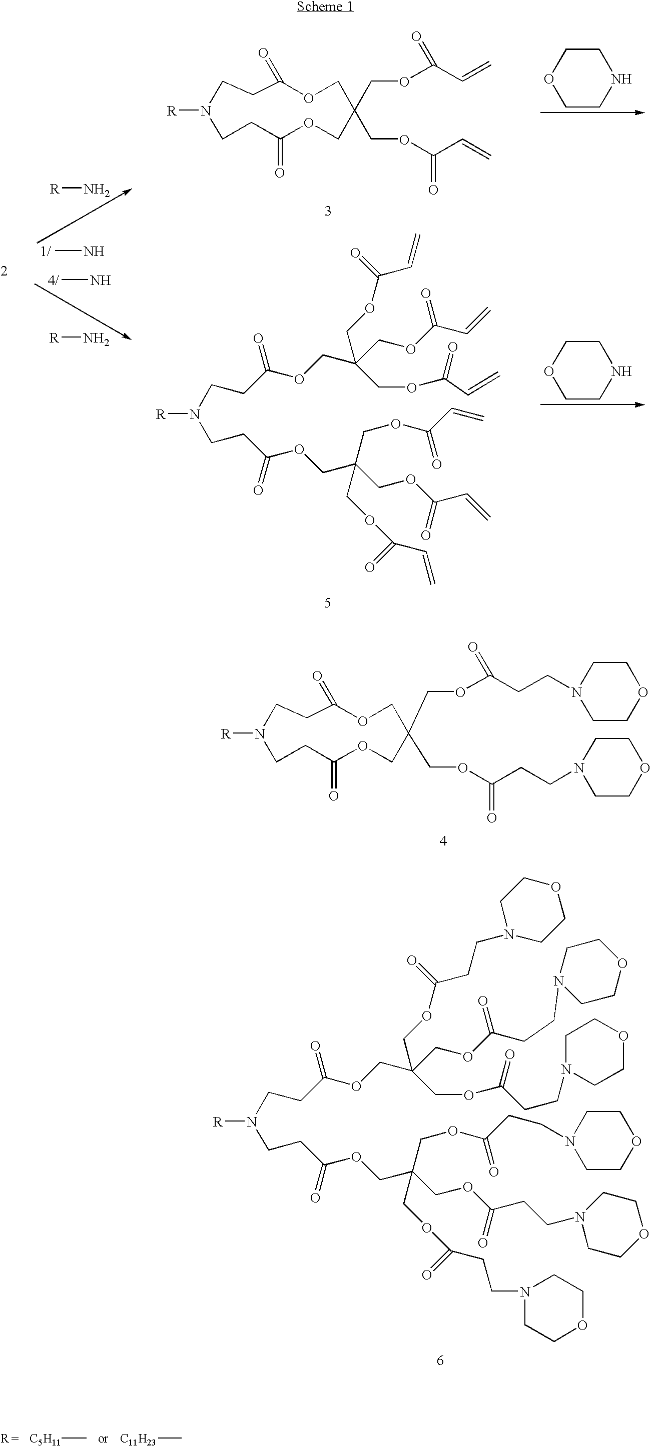

In the case of (C₅), 0.5 mole of [1] or [2] per —NH produced a looped macrocyclic-mono-dendron type product [3] in high yield. Using a 4 mole per —NH excess of monomer reagent gave primarily an ideal, di-dendron dendrimer [5]; whereas, a slightly lower excess of acrylate monomer (e.g., 1 mole (Aₓ):core-NH) produced mixtures of [3] and [5]. (See Scheme 1 above.)

This suggests that the first order intramolecular looping reaction can become competitive under these conditions with the second order, bimolecular reaction to produce ideal di-dendron products if the excesses are reduced even slightly. All products were isolated by size exclusion column chromatography workup (i.e., Sephadex™ column) and characterized by ¹H, ¹³C-NMR, FT-IR, HPLC, MALDI-TOF (see Examples below). Performing a ninhydrin test or allowing these products to react with smaller electrophilic reagents such as methyl acrylate or phenyl isothiocyanate indicated there were no detectable active hydrogens in these mono- and di-dendron products. In each case the acrylate terminated products were pacified to avoid polymerization by conversion into morpholine terminated products [4] and [6], respectively.

### Linear-α,ω-Alkyleneamine Cores

Linear-α,ω-alkyleneamine cores possessing from 2-12 methylene spacer linkages were added to slight or modest excess of the branched TMPTA or PTA reagents in methanol (i.e., 1.25-4 (Aₓ) molar excess:core-NH). A range of different products was obtained as a function of the acrylate/core-NH ratio. Using a low (Aₓ):core-NH ratio of 1.25 produced polymeric/gel-like products from all of the α,ω-alkyleneamine cores, with the exception of EDA. For example, reducing the core spacer linkage to two methylene groups (i.e., EDA) under identical conditions provided a nearly quantitative yield of tri-dendron adduct [7], using either TMPTA [1] or PTA [2] (see Scheme 2 below).

Using branched acrylate monomer TMPTA [1] in concentration ranges from 1.25-4:core-NH produced high yields of only SIS-type; (EDA core); G=1; [dendri-poly(ester-acrylate)₉] dendrimer in one step. The EDA core also yielded a tri-dendron, (EDA-core);G=1; [dendri-poly(ester-acrylate)₁₂] dendrimer [7] with PTA [2] under identical conditions. (See Scheme 2 below.)

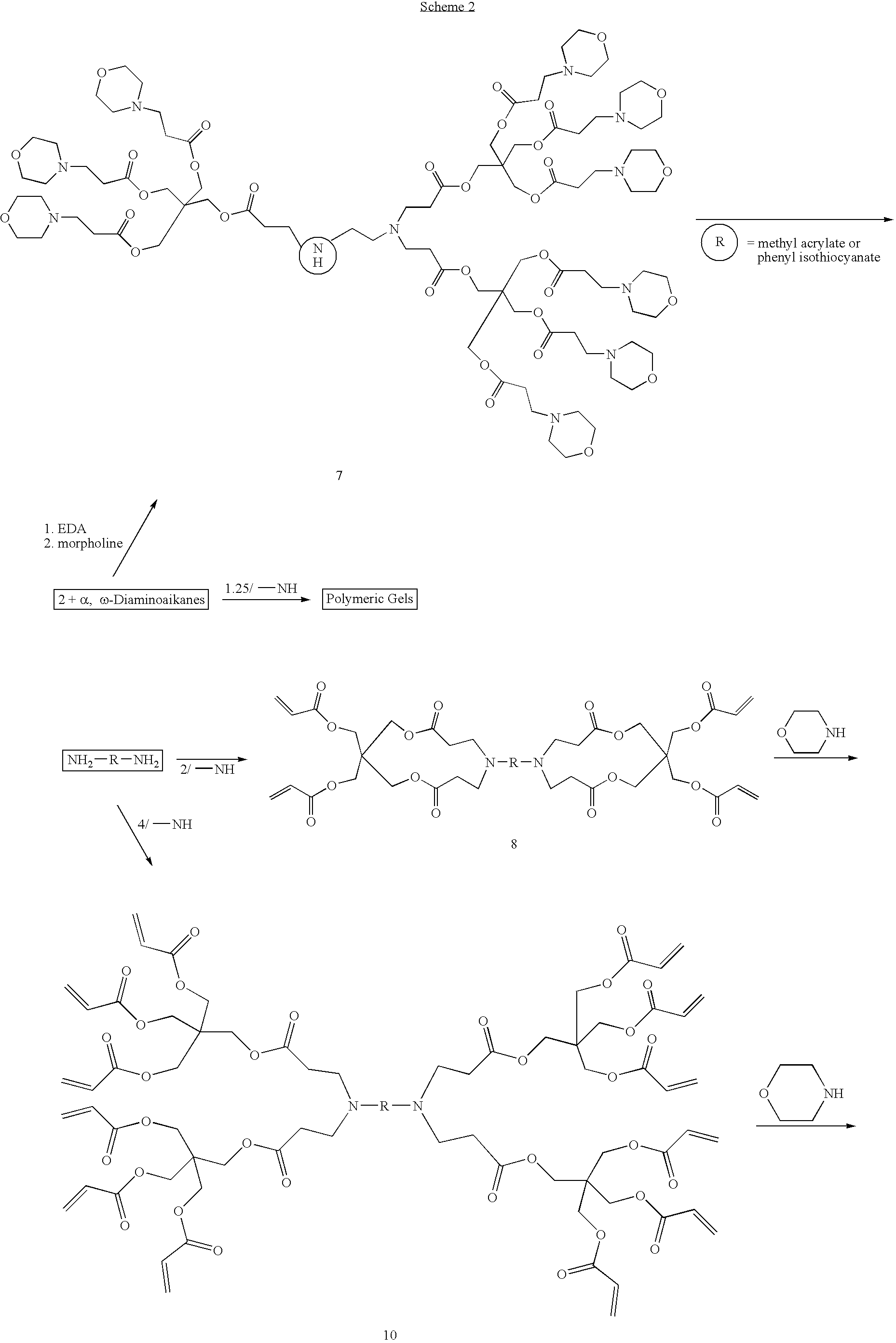

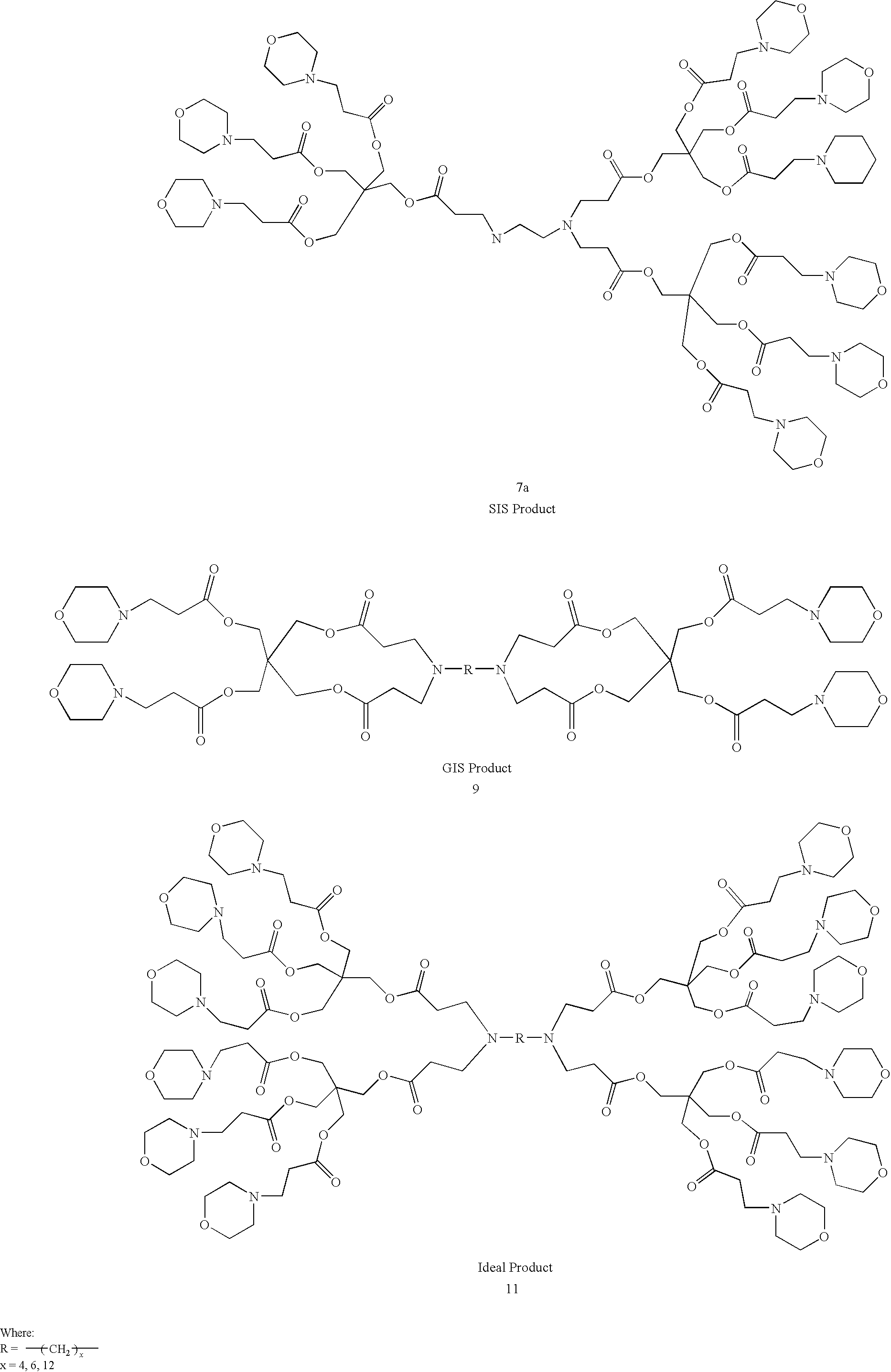

These crude reaction products were isolated from the slight excess of acrylate reagent as a colorless oil by solvent phase separation from methanol and further purified by SEC (i.e., Sephadex™ column). Increasing the (Aₓ)/NH ratio to 2.0 and 4.0, respectively, with the EDA core consistently produced the tri-dendron, SIS-type; (EDA-core); G=1; [dendri-poly(ester-morpholine)_{9/12}] dendrimer product [7] in very high yield. Using branched acrylate monomers [1] or [2] in concentration ranges from 1.25-4:core-NH produced high yields of only the tri-dendron type adduct [7] as determined by material balance, MALDI-TOF and NMR. In all cases a positive ninhydrin test was noted, indicating the presence of an active amine hydrogen supporting a sub-stoichiometric SIS-type product. Presumably such core-NH was not sterically accessible to the nano-sized branched acrylate reagents [1] or [2] to give exhaustively alkylated, ideal tetra-dendron products under these conditions. Further confirmation of the SIS-type products was garnered by subsequent reaction of the presumed tri-dendron adducts with methyl acrylate or phenyl isothiocyanate. In each case the expected products [7a] were formed by the reaction of core amine hydrogens accessible only to these small, sub-nanoscale reagents, as determined by ¹H, ¹³C-NMR and a negative ninhydrin test. As a control, EDA was combined with sub-nanoscale, linear methyl acrylate monomer under identical conditions.

Simply increasing the (Aₓ):—NH ratio to 2.0 for the less congested diamine cores (i.e. butane, hexane and dodecane) gave GIS-type, looped-di-dendron products [8] and [9] rather than polymeric gels. However, increasing the (Aₓ)/NH ratio to 4.0 produced high yields of ideal, tetra-dendron products analogous to [10]; core:diamine; G=1; [dendri-poly(ester-acrylate)₁₂] dendrimer type products in all cases with the exception of EDA. Both the presumed GIS-type looped products [8] and [9] as well as the exhaustively alkylated, ideal dendrimers [10] and [11] tested negative when exposed to ninhydrin. All these acrylate terminated dendrimers, such as [8] and [10], were predisposed to undergo polymerization if stored unprotected at room temperature. However, by shielding from exposure to light with aluminum foil, samples could be stored for short intervals at low temperature. Room temperature, shelf stable products were readily obtained by adding a nucleophilic agent such as: morpholine, 2-aminoethanol or 2-mercaptoethanol. When morpholine was used as the nucleophilic agent, it gave the (diamine core); G=1; [dendri-poly(ester-(morpholine)₁₂)] dendrimers, [11]. These more stable products could be further purified by size exclusion column chromatography (SEC) and were characterized ¹³C-NMR, FT-IR, HPLC, PAGE, and MALDI-TOF (see following Examples). Characterization indicated that a variety of SIS, GIS as well as ideal products were obtained which were dependent on the number of methylene linkages as well as the ratio of branched acrylate reagent [1] or [2]:core-NH.

### Branched Poly(Alkyleneamine) or Poly(Epoxy) Cores

Several poly(alkyleneamine) cores, namely: DETA [B] or TREN [A] in methanol were added to a stoichiometric excess of branched TMPTA [1] or PTA [2], i.e., 2.5 mole (Aₓ):core-NH. In each case products were isolated and immediately converted into morpholine terminated derivatives. Both [1] and [2] were found to give SIS-type products with these cores, based upon MALDI-TOF analysis. Reaction of [2] with DETA [B] and TREN [A] gave tetra-dendron [12] and penta-dendron [13] products, respectively. They are each one dendron deficient from ideal structures and should possess one active hydrogen. Although the exact position of the active hydrogen in [12] was not determined, both [12] and [13] were found to be ninhydrin positive; thus suggesting that they are SIS derived products. Further confirmation of the SIS-type products was garnered by subsequent reaction with methyl acrylate or phenyl isothiocyanate to give expected products, as determined by ¹H and ¹³C-NMR; thus supporting active hydrogen structures (see Scheme 3 below).

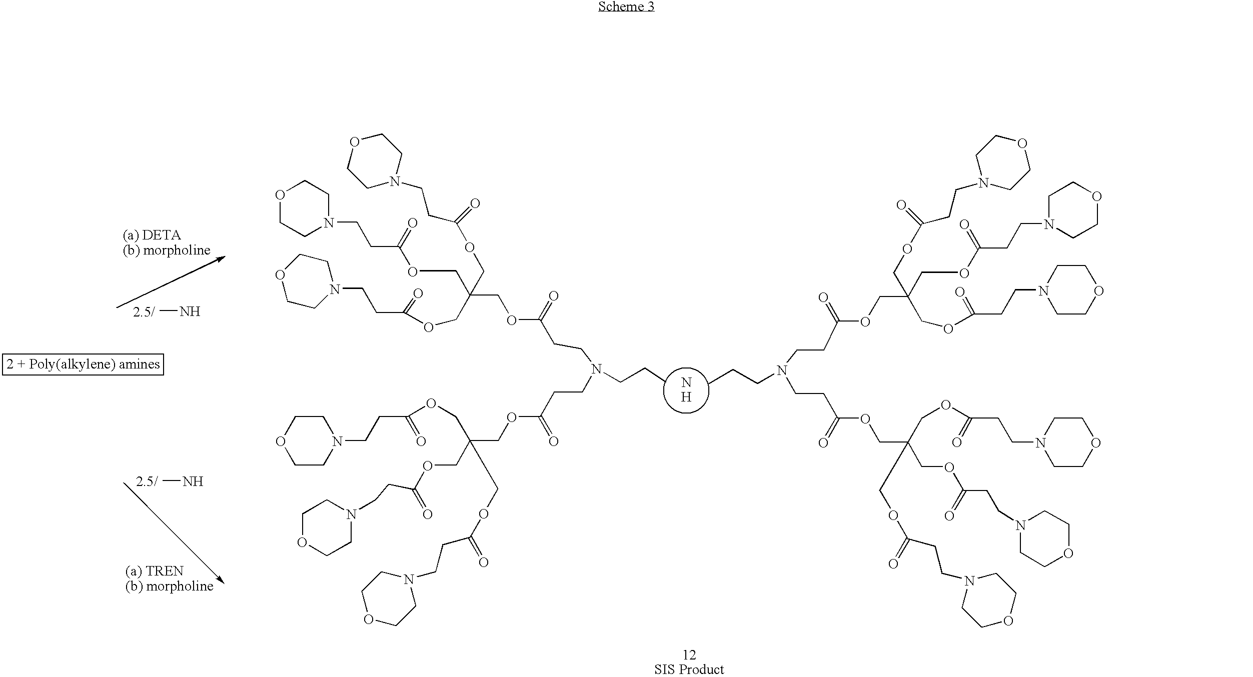

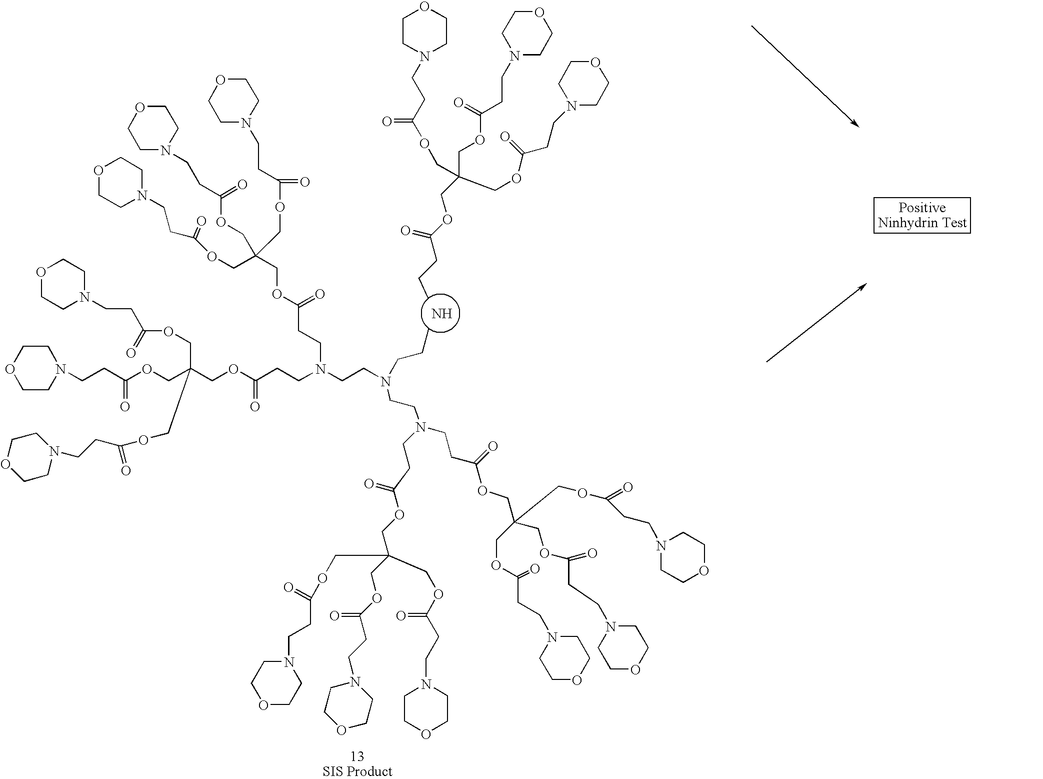

The branched poly(epoxy) core, TMPTGE [D] was reacted with TREN [A] in slight stoichiometric excess, i.e., 1.1 molar ratio per epoxy group, and the resulting macrocyclic looped product was isolated by Sephadex™ chromatography. The reaction between branched poly(epoxy) core, TMPTGE [D] and DETA [B] branching agent conducted at larger excess, i.e., 3.0 molar ratio per epoxy group, resulted in a macrocyclic, looped structure with additional free amine functionality (see Scheme 4 below).

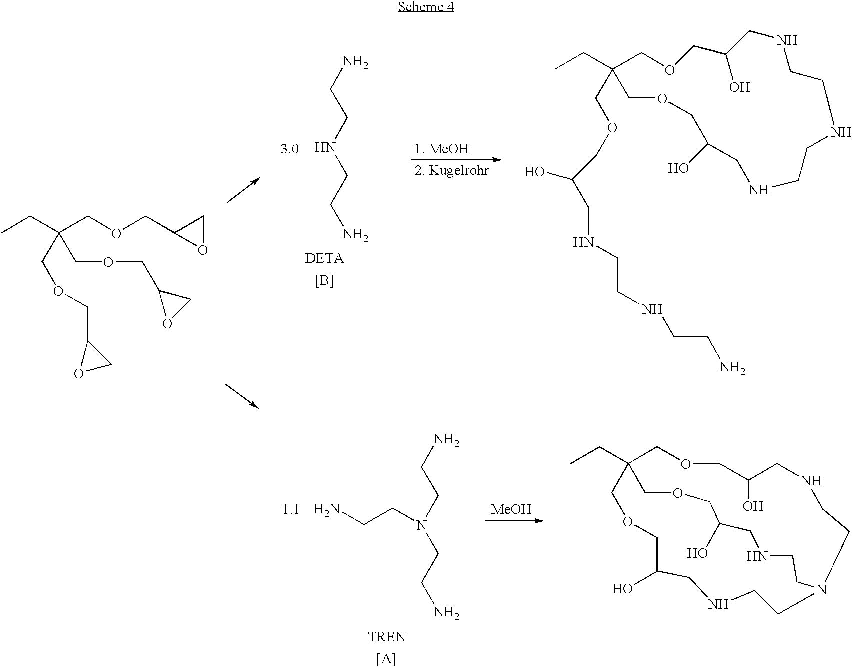

### General Methods

The reagents and solvents used were purchased from Aldrich or as indicated in the Glossary or made as described herein.

DI was made using the Millipore DI water system.

All thermometers were used without calibration.

Silica gel 60, particle size 0.040-0.063 mm, 230-400 mesh ASTM was obtained from EM Sciences.

Sephadex™ was purchased from Amersham Biosciences.

TLC was performed using Whatman Adsorption plates, 60 Å silica gel, 250 mm layer thickness.

The ¹HNMR and ¹³C NMR spectra were obtained using a Bruker WM 360 SF instrument.

MALDI-TOF mass spectrometry was performed on a Bruker Autoflex-LRF Mass Spectrometer.

HPLC spectra were measured using a Perkin Elmer (Series 200).

GPC spectra were obtained by a Waters 1515 instrument.

Poly(acrylamide) gel electrophoresis was performed on a homogeneous (15%) gel under acidic condition.

UV-Vis spectra were measured using a Hewlett Packard model 8543 and software made by Agilent Technologies.

FT-IR spectra were measured using Nicolet, MAGNA-1R-560.

The invention will be further clarified by a consideration of the following examples, which are intended to be purely exemplary of the present invention.

### Example 1

### Reaction of AmA with PTA [2] (0.5 Mole of PTA/NH) and Terminal Conversion with Morpholine

PTA [2] (3.52 g, 10 mmol) was dissolved in MeOH (5.0 mL) and cooled to 4° C. with an ice-water bath. A solution of AmA (872 mg, 10 mmol in 10 mL of MeOH) was added during a period of 5 mins. After addition, reaction was stirred at 4° C. for 30 mins. and warmed to RT. The reaction was then stirred at RT in the dark overnight. A layer of oil phased out at the bottom of the flask.

MALDI-TOF: C₂₂H₃₃NO₈ (looped, mono-dendron structure [3]) Calc: 439.50; found 440.13 (M+H), 472.19 (M+K) amu.

A solution of morpholine (4.18 g, 48 mmol) in 10 mL of MeOH was added to the reaction mixture while cooled to 4° C. After the addition, the mixture was allowed to warm to RT and stirred overnight. Then solvent was removed to give the crude product as a clear oil. Then 0.8 g of the crude was purified by Sephadex™ G (LH-20) column using MeOH as solvent. Fractions of 6-18 were combined. The spectra are as follows:

MALDI-TOF: C₃₀H₅₁N₃O₁₀ Calc. 613.74; found 631.32 (M+Na) 647.40 (M+K) amu and some other higher mass peaks; and

¹H NMR (CDCl₃, 300 MHz, δ ppm): 0.91 (t, J=7.2 Hz, 3H, —CH₃), 1.2-1.5 (m, 6H, —CH₂CH₂CH₂—), 2.43 (m, 8H), 2.56 (m, 8H), 2.65 (m, 8H), 3.55-3.61 (m, 8H), 4.23 (m, 8H); and

¹³C NMR (CDCl₃, 75 MHz, δ ppm): 13.30, 22.49, 26.56, 29.52, 31.14, 31.45, 31.88, 49.11, 50.91, 53.33, 53.62, 53.91, 53.94, 61.15, 62.05, 66.47, 173.17, 173.58.

### Example 2

### Dendronization of AmA with PTA [2] (1.0 Mole of PTA/NH) and Pacified with Morpholine

PTA [2] (7.05 g, 20 mmol) was dissolved in MeOH (10 mL) and cooled to 4° C. with an ice-water bath. The solution of AmA (872 mg, 10 mmol) in 10 mL of MeOH was added during a period of 5 mins. After the addition, the reaction was stirred at 4° C. for 30 mins. and allowed to warm to RT. The reaction was then stirred at RT in the dark overnight.

MALDI-TOF: C₂₂H₃₃NO₈ (looped, mono-dendron; structure [3]), Calc. 439.50; found 440.13 (M+H), 472.19 (M+K) amu and C₃₉H₅₃NO₁₆ (ideal, di-dendron, structure [5]), Calc. 791.84; found 792.41 (M+H) amu.

A solution of morpholine (8.36 g, 96 mmol) in 10 mL of MeOH was added to the reaction mixture while cooled to 4° C. After the addition, the mixture was allowed to warm to RT and stirred for overnight. Then solvent was removed to give the crude product as a clear oil. Then 0.8 g of the crude was purified by Sephadex™ G (LH-20) column using MeOH as solvent. Fractions of 6-18 were combined. The spectra are as follows:

¹HNMR (CD₃OD, 300 MHz, δ ppm) 0.95 (t, J=7.2 Hz, 3H, —CH₃), 1.22-1.57 (m, 6H, —CH₂CH₂CH₂—), 2.45 (m), 2.57 (m), 2.68 (m), 3.55-3.71 (m), 4.22 (mH); and

¹³CNMR (CD₃OD, 75 MHz, δ ppm) 13.33, 22.59, 26.54, 29.43, 31.67, 31.23, 49.33, 50.34, 53.37, 53.67, 53.02, 53.43, 61.12, 62.93, 66.73, 173.22, 173.61.

### Example 3

### AmA with PTA [2] (4.0 moles PTA/NH) Quenched with Morpholine

PTA [2] (7.05 g, 20 mmol) was dissolved in MeOH (10 mL) and cooled to 4° C. with an ice-water bath. The solution of AmA (218 mg, 2.5 mmol in 5 mL of MeOH) was added during a period of 5 mins. After the addition, the reaction was stirred at 4° C. for 30 mins. and allowed to warm to RT. The reaction was then stirred at RT in the dark overnight.

MALDI-TOF: C₃₉H₅₃NO₁₆ Calc. 791.84; found 792.40 (M+H) amu (ideal, di-dendron, structure [5].

A solution of morpholine (8.36 g, 96 mmol) in MeOH (10 mL) was added to the reaction mixture while cooled to 4° C. After the addition, the mixture was allowed to warm to RT and stirred overnight. Then solvent was removed to give the crude product as a clear oil. Then 0.8 g of the crude was purified by Sephadex G (LH-20) column using MeOH as solvent. Fractions of 8-16 were combined. The spectra are as follows:

MALDI-TOF: C₆₃H₁o7N₇O₂₂ Calc. 1314.56; found 1314.78 (M+H) amu (structure [6], and some other higher unidentified peaks; and

¹HNMR (CDCl₃, 300 MHz, δ ppm) 0.877 (t, J=7.5 Hz, 3H, —CH₃), 1.21-1.39 (m, 6H, —CH₂CH₂CH₂—), 2.43 (m, 24H), 2.50 (m, 16H), 2.63 (m, 16H), 3.66 (m, 24H), 4.14 (m, 16H); and

¹³CNMR (CDCl₃, 75 MHz, δ ppm) 14.31, 22.82, 26.95, 29.79, 32.22, 34.74, 49.16, 53.63, 54.10, 54.29, 62.14, 62.63, 67.06, 171.92, 172.02, 172.28.

### Example 4

### Undecylamine with PTA [2] (0.5 Mole PTA:—NH); Structure [3]

PTA [2] (3.21 g, 9.1 mmol) was dissolved in 5.0 mL of MeOH and cooled to 4° C. with an ice-water bath. The solution of undecylamine (1.56 mg, 9.1 mmol) in 10 mL of MeOH was added during a period of 5 mins. After the addition, the reaction was stirred at 4° C. for 30 mins. and allowed to warm to RT. The reaction was then stirred at RT in the dark overnight. A wax like, polymeric solid phased out at the bottom of flask. The solution was checked by MALDI-TOF and has the following spectra:

MALDI-TOF: C₂₈H₄₅NO₈ (looped, mono-dendron; structure [3]) Calc. 523.66; found, 524.28 (M+H), 556.33 (M+K) amu.

### Example 5

### Undecylamine with PTA [2] (1.0 mole PTA:—NH); Structures [3] and [5]

PTA [2] (6.42 g, 18.2 mmol) was dissolved in 10 mL of MeOH and cooled to 4° C. with an ice-water bath. A solution of undecylamine (1.56 mg, 9.1 mmol) in 10 mL of MeOH was added over a period of 5 mins. After addition, the reaction was stirred at 4° C. for 30 mins. and allowed to warm to RT. The reaction was then stirred at RT overnight in the dark (protected from light). The spectra are as follows:

MALDI-TOF: C₂₈H₄₅NO₈ (looped, mono-dendron; structure [3]) Calc. 523.66; found, 524.28 (M+H), 556.33 (M+K) amu and C₄₅H₆₅NO₁₆ (di-dendron; structure [5]) Calc. 875.99; found, 876.51 (M+H) amu.

A solution of morpholine (7.61 g, 87.4 mmol) in 10 mL of MeOH was added to the reaction mixture while cooling at 4° C. After addition, the mixture was allowed to warm to RT and stirred overnight. The solvent was removed to give the crude product as a clear oil, yield 14.2 g (95%).

### Results of Examples 1-5:

The results of Examples 1-5 are summarized in Table 1. Numbers in parenthesis refer to structures displayed in Scheme 1. The PTA per NH ratio defines the structures formed.

TABLE 1

| (A)ₓ:NH) | Main Product | Product Type |
|---|---|---|
| 0.5:1 | looped, mono-dendron | GIS |
| | (3) or (4) | |
| 1:1 | mixture of (3) and (5) | GIS/Ideal |
| | or (4) and (6) | |
| 4:1 | di-dendron (5) or (6) | Ideal/di-dendron |

### Example 6

### Preparation of [EDA]; (G=1); Dendri{CH₂—CH₂—CO₂—CH₂C(Et)-(CH₂CO₂CH═CH₂)₂}₂; Tri-Dendron Precursor to Structure [1]

To a 100 mL round-bottom flask with a stir bar was added TMPTA [1] (29.6 g, 0.10 mol) in 15 mL of MeOH and cooled to ˜4° C. EDA (1.2 g, 0.02 mol) in 5 mL of MeOH was added over 5 mins. This mixture was stirred at 30° C. for 18 hours. The mixture was cooled to 20° C. and poured into 150 g of stirred MeOH. The product phased out by allowing the mixture to stand without stirring for 1 hour. The MeOH layer was decanted and this process was repeated two more times. This clear, viscous mixture was evacuated at high vacuum for 3 hours while protecting it from light using aluminum foil to give 20 g (100% based on tri-adduct product and 80% based on tetra-adduct product) of material indicating that most of the material was the tri-dendron adduct consisting of three TMPTA's to one EDA. Its spectra are as follows:

A MALDI-TOF mass spectrum of this product indicated a major peak at 950 daltons corresponding to a tri-dendron adduct with theoretical MW=949 daltons, tri-dendron precursor to structure [1]. A very small peak at 1245 daltons was observed for the tetra-dendron adduct; and

¹³C-NMR (500 MHz, CDCl₃) δ 7.45, 23.00, 23.14, 32.38, 40.77, 40.86, 49.48, 63.88, 64.05, 128.04, 131.26, 165.69, 172.10.

### Example 7

### Preparation of [HDA]; (G=1); Dendri{CH₂—CH₂—CO₂—CH₂C(Et)-(CH₂CO₂CH═CH₂)₂}; Tetra-Dendron, Structure [10]

To a 100 mL round-bottom flask containing a stir bar was added TMPTA [1] (29.6 g, 0.10 mol) and 10 mL of MeOH. To this mixture cooled at 4° C. was added HDA (2.32 g, 0.02 mol) in 20 mL of MeOH. This mixture was heated at 30° C. for 18 hours under N₂. This mixture was cooled to ±15° C. and poured into 150 mL stirred MeOH. The product phased out by allowing this mixture to stand without stirring for 1 hour and protecting the flask from light by wrapping the flask with aluminum foil. The MeOH layer was decanted and this process was repeated two more times to give a colorless, clear and viscous liquid. This mixture was evacuated at high vacuum for 3-5 hours to give 24 g (92% yield based on ideal, tetra-dendron). A MALDI-TOF mass spectrum of this product exhibited a peak at 1301 daltons for the tetra-dendron product and several decomposition peaks derived from the tetra-dendron product.

### Example 8

### Conversion of [HDA]; (G=1); Dendri{(CH₂—CH₂—CO₂—CH₂C(Et)-(CH₂OC═OCH═CH₂)₂}₄ to the Morpholine Terminated Adduct; Structure [11]

To a 250 mL round-bottom flask containing a stir bar was added poly(esteramine), G=1, hexamethylenediamine core (24 g, 18.4 mmol, 147 mmol acrylate; prepared in Example 7) in 50 mL of DME. To this mixture cooled to ˜4° C. was added morpholine (14 g, 160 mmol, 1.1 mole per acrylate) in 50 mL of DME over about 5-10 mins. This mixture was warmed to RT and stirred for 24 hours. This mixture was stripped of volatiles on a rotary evaporator and high vacuum at 30° C. for 18 hours to give 34 g (94% yield) of product. A MALDI-TOF mass spectrum of this material showed a peak corresponding to the theoretical molecular weight of 1998 amu along with several lower peaks derived from fragmentation of the 1998 peak. A ¹³C NMR spectrum of this material shows the product is very clean and with the correct number of carbons for the desired product. Its spectra are as follows:

¹³C NMR (500 MHz, CDCl₃): 7.42, 22.82, 27.21, 27.54, 32.15, 40.78, 40.89, 48.97, 53.40, 53.94, 59.04, 63.56, 66.85, 71.79, 171.86, 172.16.

### Example 9

### Conversion of [HDA]; (G=1);Dendri{CH₂—CH₂—CO₂—CH₂C(Et)-(CH₂CO₂CH═CH₂)₂}₄ to the Ethanolamine Terminated Adduct

To a 250 mL round-bottom flask containing a stir bar was added ethanolamine (27 g, 442 mmol, 3 equiv. per acrylate) in 50 mL of DME. To this mixture cooled at 4° C. was added hexamethylenediamine core polyesteramine, octa-acrylate (24 g, 18.4 mmol, 8 moles acrylate per dendrimer) in 50 mL of DME dropwise over about 10 mins. This mixture was stirred at 25° C. for 2 days under N₂. The mixture was stripped of volatiles with a rotary evaporator. This crude material was poured into a rapidly stirred ethyl acetate. After a few minutes of stirring, the mixture was allowed to stand for 1 hour to allow separation of the two layers and the ethyl acetate layer was decanted off. The same volume of ethyl acetate was added, the mixture rapidly stirred and separate as before. This was repeated a second time for a total of three washes. The clear, colorless viscous oil was evacuated at high vacuum overnight at RT to give 29.7 g (90%) of the desired product. An analysis by PAGE on a 15% crosslinked homogeneous poly(acrylamide) gel using PAMAM dendrimers as standards (G=2-6) indicated material that was a sharp, tight band corresponding to a G=1 PAMAM dendrimer.

### Example 10

### Conversion of [EDA]; (G=1); Dendri{CH₂—CH₂—CO₂—CH₂C(Et)-(CH₂CO₂CH═CH₂)₂}₄ to the Mercaptoethanol Terminated Adduct

To a 250 mL round-bottom flask with a stir bar was added the ethylenediamine core polyesteramine, G=1, hexa-acrylate (19 g, 20 mmol, 120 mmol acrylate) in 50 mL of DME and mercaptoethanol (10.4 g, 132 mmol, 1.1 equiv. per acrylate) in 20 mL of DME. This mixture was stirred for 2 days at RT. This mixture was stripped of volatiles on a rotary evaporator. The resulting material was mixed with 150 mL of ethyl acetate and rapidly stirred with a stir bar. This heterogeneous mixture was allowed to settle for about 1 hour. The clear ethyl acetate layer was decanted. This process was repeated two more times. A PAGE of this material on a 15% crosslinked homogeneous poly(acrylamide) gel with PAMAM dendrimer standards G=2-6, indicated a sharp, tight band corresponding to a G=1 PAMAM dendrimer.

### Example 11

### Preparation of [EDA]; (G=1); Dendri{CH₂—CH₂CH₂—CH₂C—(CH₂CO₂CH═CH₂)₃}₃; (1.25 Moles of PTA Per —NH)

To a 50 mL round-bottom flask with a stir bar was added PTA [2] (17.6 g, 0.50 mmol) and 15 mL of MeOH cooled to ˜4° C. EDA (600 mg, 10 mmol) in 10 mL of MeOH was added over about 5 mins. This mixture was stirred at 30° C. for 18 hours. The crude reaction product was cooled to 20° C. and poured into 150 g of stirred MeOH. A clear/cloudy product layer phased out by allowing the mixture to stand without stirring for hour. The MeOH layer was decanted and this process was repeated two more times. A clear, viscous product residue was devolatilized at high vacuum for 3 hours while protecting from light using aluminum foil to give 9 g (95% based on a tri-dendron product). A MALDI-TOF mass spectrum of this product indicated a major peak at 1117 amu (M+H) supporting the tri-dendron product which has a theoretical MW=1116 amu; precursor to structure [1].

### Example 12

### Conversion of [EDA];(G=1); Dendri{CH₂—CH₂—CO₂—CH₂C—(CH₂OCO₂CH═CH₂)₃}₃ to the Morpholine Adduct

To a 100 mL round-bottom flask containing a stir bar was added the [EDA core]; (G=1) poly(acrylate)₉ dendrimer (9 g, 8.1 mmol, 73 mmol acrylate) in 20 g of MeOH and the resulting mixture was cooled at ˜4° C. To this mixture was added dropwise over ˜5 mins. morpholine (9.5 g, 110 mmol, 1.5 moles per acrylate) in 20 g of MeOH at 25° C. This mixture was stirred at 4° C. for 30 mins. then at RT for 18 hours. The volatiles were removed on a rotary evaporator followed by high vacuum to give 19 g of crude material. This crude material was mixed with 150 mL of MeOH and heated to ˜60° C. to make the solution homogeneous. Allowing this solution to cool to RT over ˜4 hours, caused a clear, oily product to phase out as an immiscible layer. This process was repeated two more times to produce a morpholine free product that weighed, 16 g (92% yield). Its spectra are as follows:

¹H NMR (300 MHz, CDCl₃): δ 2.41 (bs, 36H), 2.47-2.50 (bs, 30H), 2.60-2.62 (bs, 18H), 2.73 (bs, 13H), 3.41 (bs, 4H), 3.54 (bs, 6H), 3.63 (bs, 36H), 3.8-4.2 (bm, 29 H); and

¹³C NMR (75 MHz, CDCl₃): δ 32.22, 42.65, 44.00, 49.48, 53.61, 54.09, 54.26, 62.09, 62.60, 66.84, 66.93, 67.02, 171.90, 172.10, 172.20.

### Example 13

### Preparation of [EDA]; (G=1); Dendri{CH₂—CH₂—CO₂—CH₂C—(CH₂CO₂CH═H₂)₃}₃; (4 Moles PTA per NH) and Conversion to the Morpholine Terminated Adduct

To a 50 mL round-bottom flask equipped with a stir bar was added PTA [2] (18.7 g, 53.1 mmol) in 15 mL of MeOH and cooled to ˜4° C. EDA (200 mg, 3.3 mmol) in 10 mL of MeOH was added over a 5 min. period. This mixture was stirred at 30° C. for 18 hours. This mixture was cooled to 4° C. and added dropwise to morpholine (27 g, 318 mmol, 1.5 moles per acrylate) in 10 g of MeOH, followed by stirring at RT for 24 hours under N₂. The volatiles were removed with a rotary evaporator followed by high vacuum to give 37.5 g of crude material. An aliquot of this mixture (836 mg) was purified using a Sephadex™ LH-20 column (void volume 105 mL) in MeOH taking 40×3 mL fractions. Product was collected in fractions 1-18 to give 140 mg (5.7 g product, 90% yield, as the (3:1), tri-dendron adduct). Its spectra are as follows:

¹H NMR (300 MHz, CDCl₃): δ 2.41 (bs, 36H), 2.47-2.50 (bs, 30H), 2.60-2.62 (bs, 18H), 2.73 (bs, 13H), 3.41 (bs, 4H), 3.53 (bs, 6H), 3.63 (bs, 36H), 3.8-4.2 (bm, 29 H); and

¹³C NMR (75 MHz, CDCl₃): δ 32.21, 42.65, 44.00, 49.48, 53.59, 54.09, 54.25, 62.08, 62.60, 66.91, 67.00, 171.85, 171.96, 172.22.

### Results of Examples 11-13:

The results of Examples 11-13 are summarized in Table 2. Numbers in parenthesis refer to a structure displayed in Scheme 2. SIS leads to the formation of EDA tri-dendron (7) independently of the PTA per NH ratio.

TABLE 2

| (A)ₓ:NH) | Main Product | Product Type |
|---|---|---|
| 1.25:1 | tri-dendron (7) | SIS |
| 2:1 | tri-dendron (7) | SIS |
| 4:1 | tri-dendron (7) | SIS |

### Example 14

### Reaction of [EDA]; (G=1); Dendri{CH₂—CH₂CO₂—CH₂C—(CH₂CO₂CH═CH₂)₃}₃; Morpholine Terminated Adduct with Methyl Acrylate; Structure [7a]

To a 10 mL round-bottom flask with a stir bar was added polyesteramine dendrimer, G=1, EDA core, morpholine terminated adduct (150 mg, 7.9×10⁻⁵ mol) and 2 g of MeOH. To this mixture was added methyl acrylate (50 mg, 5.8×10⁻⁴ mol). The flask was sealed with a polypropylene cap and heated at 40° C. for 24 hours. This mixture was cooled and evacuated on a rotary evaporator followed by high vacuum at 40° C. for 4 hours to give 158 mg material. Its spectra are as follows:

¹H NMR (300 MHz, CDCl₃): δ 2.40 (bs, 36H), 2.46-2.56 (bs, 30H), 2.58-2.67 (bs, 18H), 2.72 (bs, 13H), 3.39 (bs, 4H), 3.53 (bs, 6H), 3.63 (bs, 36H), 3.98-4.2 (bm, 29 H); and

¹³C NMR (75 MHz, CDCl₃): δ 32.04, 32.18, 42.51, 42.63, 43.81, 43.97, 49.69, 51.88, 53.58, 54.07, 54.23, 62.08, 62.58, 66.91, 63.08, 66.89, 66.99, 67.08, 171.89, 171.96, 172.22.

### Example 15

### Reaction of [EDA]; (G=1); Dendri{CH₂—CH₂CO₂—CH₂C—(CH₂CO₂CH═CH₂)₃}₃, Morpholine Terminated Adduct with Phenyl Isothiocyanate; Structure [7a]

To a 10 mL round-bottom flask with a stir bar was added polyesteramine dendrimer, G=1, EDA core, morpholine terminated adduct (200 mg, 1.0×10⁻⁴ mol) and 2 g of methylene chloride. To this mixture was added phenyl isothiocyanate (50 mg, 5.8×10⁻⁴ mol, 6 moles). The reaction was stirred at RT for 24 hours, devolatilized on a rotary evaporator followed by high vacuum at 40° C. for 1 hour to give 250 mg of material. This crude product was warmed in 2 g of MeOH to give a clear solution. Cooling the solution to 25° C. caused an immiscible layer to phase out. This procedure was repeated three times to yield an oily product which devolatilized at 40° C. for 3 hours to give 220 mg of product. This material, according to NMR analysis, was consistent with a thiourea structure that would be expected by reaction with an amine core, active hydrogen. Its spectra are as follows:

¹H NMR (300 MHz, CDCl₃): δ 2.42 (bs, 36H), 2.47-2.58 (bs, 30H), 2.60-2.70 (bs, 18H), 2.74 (bs, 13H), 3.42 (bs, 4H), 3.54 (bs, 6H), 3.63 (bs, 36H), 3.8-4.2 (bm, 29H), 7.15-7.36 (m, 6H); and

¹³C NMR (75 MHz, CDCl₃): δ 32.21, 42.65, 44.00, 49.67, 53.93, 53.60, 54.08, 54.25, 62.07, 62.62, 63.09, 66.99, 125.96, 127.53, 128.76, 129.76, 171.91, 171.96, 172.27.

### Example 16

### Preparation of [1,4-Diaminobutane]; (G=1); Dendri{CH₂—CH₂—CO₂—CH₂C—(CH₂CO₂CH═CH₂)₃}₄ (2 Moles PTA Per NH) and Conversion to Morpholine Terminated Adduct

To a 50 mL round-bottom flask containing a stir bar was added PTA [2] (11.7 g, 32 mmol, 2 moles per NH) and 10 mL of MeOH. To this mixture, cooled to 4° C., was added 1,4-diaminobutane (365 mg, 4.1 mmol) in 5 mL of MeOH. This reaction mixture was stirred at 25° C. for 18 hours under N₂. After cooling to ˜15° C., morpholine (17 g, 195 mmol, 1.5 moles per acrylate) was added dropwise over ˜5 mins. into 40 mL of stirred MeOH cooled at 4° C. This mixture was stirred for 18 hours at RT. This mixture was evacuated of volatiles with a rotary evaporator followed by high vacuum for 3-5 hours to give 24.53 g of crude material. An aliquot (897 mg) was purified on a Sephadex™ LH-20 column (void volume 105 mL) in MeOH taking 30×3 mL fractions. A TLC (MeOH) indicated the product was in fractions 5-23. These collected fractions were stripped of volatiles to a constant weight to give 140 mg (4.3 g, 93% yield for a di-looped, di-dendron, structure [3]; MW=1140). Its spectra are as follows:

¹H NMR (300 MHz, CDCl₃): δ 1.32 (bs, 4H), 2.38 (bs, 36H), 2.45-2.50 (bs, 30H), 2.59 (bm, 18H), 2.69 (bm, 13H), 3.39 (bs, 4H), 3.52 (bs, 6H), 3.60 (bs, 36H), 3.9-4.2 (bm, 29H); and

¹³C NMR (75 MHz, CDCl₃): δ 24.84, 32.19, 42.60, 43.07, 43.80, 44.96, 49.15, 49.30, 53.57, 54.06, 54.24, 62.09, 62.59, 63.08, 66.79, 66.89, 67.00, 171.90, 172.10, 172.20.

### Example 17

### Preparation of [1,4-Diaminobutane]; (G=1); Dendri{CH₂—CH₂—CO₂—CH₂C—(CH₂CO₂CH═CH2)₃}₄ with 4 Moles PTA Per NH and Conversion to Morpholine Surface

To a 50 mL round-bottom flask containing a stir bar was added PTA [2] (19.0 g, 32 mmol, 4 moles per NH) and 10 mL of MeOH. To this mixture, cooled at 4° C., was added 1,4-diaminobutane (303 mg, 3.3 mmol) in 5 mL of MeOH. This mixture was stirred at 25° C. for 18 hours under N₂. This mixture was added dropwise over ˜5 mins., while stirring into 40 mL of MeOH containing morpholine (28 g, 321 mmol, 1.5 moles per acrylate). This mixture was stirred for 18 hours at RT. This crude reaction mixture devolatilized on a rotary evaporator followed by submission to high vacuum for 3-5 hours to give 37.17 g of crude material. An aliquot (2.8 g) was purified on a Sephadex™ LH-20 column (void volume 365 mL) in MeOH taking 30×8 mL fractions. A TLC (MeOH) indicated the product eluted in fractions 2-16. These collected fractions were stripped of volatiles to a constant weight giving 600 mg (8.0 g, 96% yield for a (4:1); tetra-dendron adduct with theoretical MW=2540), structure [11]. Its spectra are as follows:

¹H NMR (300 MHz, CDCl₃): δ 1.35 (bs, 4H), 2.41 (bs, 36H), 2.45-2.58 (bs, 30H), 2.59 (bm, 18H), 2.62 (bm, 13H), 3.37 (bs, 4H), 3.41 (bs, 6H), 3.52 (bm, 4H), 3.63 (bs, 36H), 4.0-4.2 (bm, 29H); and

¹³C NMR (75 MHz, CDCl₃): δ 24.84, 32.13, 42.42, 42.56, 43.03, 43.15, 43.88, 43.07, 48.92, 53.50, 54.00, 54.16, 61.99, 62.14, 62.48, 66.79, 66.89, 67.00, 171.73, 171.03, 172.06.

### Example 18

### Preparation of [HDA] (G=1); Dendri{CH₂—CH₂—CO₂—CH₂C—(CH₂CO₂CH═CH₂)₃}₄, (4 Moles PTA Per NH) and Conversion to Morpholine Terminated Adduct

To a 50 mL round-bottom flask equipped with a stir bar was added PTA [2] (18.2 g, 51.7 mmol) and 10 mL of MeOH. HDA (400 mg, 3.4 mmol) in 10 mL of MeOH was added to this mixture, which had been cooled to 4° C. This reaction mixture was stirred at 25° C. for 18 hours under N₂. After cooling to ˜15° C. this reaction mixture was added dropwise over ˜5 mins. into a stirred morpholine solution (27 g, 310 mmol, 1.5 moles per acrylate) in 40 mL MeOH cooled at 4° C. This reaction mixture was stirred for 19 hours at RT, devolatilized on a rotary evaporator, followed by submission to high vacuum for 3-5 hours to give 36 g of crude material. An aliquot (1.0 g) was purified on a Sephadex LH-20 column (void volume 105 mL) in MeOH taking 30×3 mL fractions. A TLC (MeOH) indicated the product eluded in fractions 1-16. These collected fractions were stripped of volatiles to a constant weight to give 250 mg (9 g, 97% yield of the tetra-dendron adduct). Its spectra are as follows:

¹H NMR (300 MHz, CDCl₃): δ 1.21 (bs, 4H), 1.37 (bs, 4H), 2.41 (bs, 48H), 2.46-2.7 (bm, 64H), 3.40 (bs, 4H), 3.52 (bs, 4H), 3.63 (bs, 48H), 4.0-4.2 (bs, 32H); and

¹³C NMR (75 MHz, CDCl₃): δ 27.64, 32.22, 42.61, 43.10, 44.02, 49.05, 53.60, 54.09, 54.27, 62.10, 62.60, 66.92, 67.02, 171.88, 172.22.

### Example 19

### Preparation of [HDA]; (G=1); Dendri{CH₂—CH₂—CO₂—CH₂C—(CH₂CO₂CH═CH₂)₃}₄ (2 Moles PTA Per NH) and Conversion to Morpholine Terminated Adduct

To a 50 mL round-bottom flask containing a stir bar was added PTA [2] (9.7 g, 27.6 mmol) and 10 mL of MeOH. To this mixture, cooled at 4° C., was added HDA (400 mg, 3.4 mmol) in 10 mL of MeOH. This mixture was stirred at 25° C. for 18 hours under N₂. Then the mixture was cooled to ˜15° C. and added dropwise over ˜5 mins. into 40 mL of stirred MeOH containing morpholine (14.4 g, 165 mmol, 1.5 moles per acrylate) cooled at 4° C. This mixture was stirred for 19 hours at RT, devolatilized on a rotary evaporator, followed by submission to high vacuum for 3-5 hours to give 22 g of crude material. An aliquot (1.0 g) was purified on a Sephadex™ LH-20 column (void volume 105 mL) in MeOH taking 30×3 mL fractions. A TLC (MeOH) indicated the product eluded in fractions 1-16. These collected fractions were stripped of volatiles to a constant weight to give 250 mg (5.5 g, 89% yield of the tri-dendron adduct). Its spectra are as follows:

¹H NMR (300 MHz, CDCl₃): δ 1.20 (bs, 4H), 1.35 (bs, 4H), 2.39 (bs, 48H), 2.43-2.8 (bm, 64H), 3.40 (bs, 4H), 3.52 (bs, 4H), 3.61 (bs, 48H), 4.0-4.2 (bs, 32H); and

¹³C NMR (75 MHz, CDCl₃): δ 27.64, 32.22, 42.61, 43.10, 44.02, 49.05, 53.60, 54.09, 54.27, 62.10, 62.60, 66.92, 67.02, 171.88, 172.22.

### Example 20

### Preparation of [1,12-Diaminodecane]; (G=1); Dendri{CH₂—CH₂—CO₂—CH₂C—(CH₂CO₂CH═CH₂)₃}₄, (2 Moles PTA Per NH) and Conversion to Morpholine Terminated Adduct

To a 50 mL round-bottom flask containing a stir bar was added PTA [2] (17.6 g, 50 mmol, 2.2 moles per NH) and 10 mL of MeOH. To this mixture cooled at 4° C. was added 1,12-diaminododecane (1.16 g, 5.8 mmol) in 20 mL of MeOH. This mixture was stirred at 25° C. for 18 hours under N₂. This mixture was added dropwise over ˜5 mins. to a mixture of 40 g of MeOH containing morpholine (26 g, 300 mmol, 1.5 moles), cooled at 4° C. This mixture was stirred at RT for 18 hours, devolatilized on a rotary evaporator followed by submission to high vacuum for 3-5 hours to give 37.5 g. An aliquot (1.0 g) was purified on a Sephadex™ LH-20 column (void volume 105 mL) in MeOH taking 30×3 mL fractions. A TLC (MeOH) indicated the product eluded in fractions 1-16. These collected fractions were stripped of volatiles to a constant weight to give 146 mg (5.5 g, quantitative yield) of the di-dendron adduct containing two loops, with theoretical MW=1252, structure [9]. Its spectra are as follows:

¹H NMR (300 MHz, CDCl₃): δ 1.20 (bs, 14H), 1.38 (bs, 4H), 2.40 (bs, 24H), 2.41-2.55 (bs, 18H), 2.55-2.65 (bs, 10H), 2.68-2.78 (bs, 6H), 3.39 (bs, 4H), 3.52 (bs, 2H), 3.62 (bs, 24H), 4.0-4.2 (bs, 18H); and

¹³C NMR (75 MHz, CDCl₃): δ 27.76, 29.94, 32.20, 42.46, 42.60, 43.08, 44.03, 49.07, 49.18, 49.39, 53.58, 54.068, 54.26, 62.09, 62.59, 63.07, 67.00, 171.86, 172.16, 172.21, 172.53.

### Example 21

### Preparation of [1,12-Diaminodecane]; (G=1); Dendri{CH₂—CH₂—CO₂—CH₂C—(CH₂CO₂CH═CH₂)₃}₄, (4 Moles PTA Per NH) and Conversion to Morpholine Terminated Adduct

To a 50 mL round-bottom flask containing a stir bar was added PTA [2] (19.9 g, 50 mmol, 2.2 moles per NH) and 10 mL of MeOH. To this mixture cooled at 4° C. was added 1,12-diaminododecane (706 g, 5.8 mmol) in 20 mL of MeOH. This mixture was stirred at 25° C. for 18 hours under N₂. This mixture was added dropwise over ˜5 mins. to a mixture of 40 g of MeOH containing morpholine (29 g, 333 mmol, 1.5 moles), cooled at 4° C. This mixture was stirred at RT for 18 hours. This mixture was evacuated of volatiles with a rotary evaporator followed by high vacuum for 3-5 hours to give 39.25 g of crude product. An aliquot (2.8 g) was purified on a Sephadex™ LH-20 column (void volume 365 mL) in MeOH taking 30×8 mL fractions. A TLC (MeOH) indicated the product eluded in fractions 2-16. These collected fractions were stripped of volatiles to a constant weight to give 600 mg (94% yield of the (4:1), tetra-dendron adduct with theoretical MW=2658 daltons), structure [11]. Its spectra are as follows:

¹H NMR (300 MHz, CDCl₃): δ 1.22 (bs, 14H), 1.39 (bs, 4H), 2.41 (bs, 24H), 2.45-2.58 (bs, 18H), 2.59-2.68 (bs, 10H), 2.68-2.78 (bs, 6H), 3.41 (bs, 4H), 3.52 (bs, 2H), 3.64 (bs, 24H), 4.0-4.2 (bs, 18H); and

¹³C NMR (75 MHz, CDCl₃): δ 27.76, 29.94, 32.22, 32.29, 42.61, 42.61, 43.11, 43.23, 44.06, 49.07, 49.09, 53.60, 54.09, 54.28, 62.11, 62.24, 66.91, 67.03, 171.80, 172.18, 172.23, 172.55.

### Results of Examples 16-21:

The results of Examples 16-21 are summarized in Table 3. Numbers in parenthesis refer to structure displayed in Scheme 2. The PTA per NH ratio of 1.25:1 resulted in polymer gel formation.

TABLE 3

| (A)ₓ:NH) | Main Product | Product Type |
|---|---|---|
| 1.25:1 | Polymers, gels | |
| 2:1 | di-looped; di-dendron (8) | GIS |
| | and (9) | |
| 4:1 | tetra-dendron (10) and (11) | Ideal |

### Example 22

### Preparation of [DETA]; (G=1); Dendri{CH₂—CH₂—CO₂—CH₂C—(CH₂CO₂CH═CH₂)₃}₄, (2.8 Moles Per NH) and Conversion to Morpholine Terminated Adduct

To a 50 ml round-bottom flask with a stir bar was added PTA [2] (6 g, 0.17 mmol, 2.8 moles per NH) and 15 mL of MeOH. To this mixture cooled at ˜4° C. was added DETA [B] (119 mg, 1.2 mmol) in 10 mL of MeOH over about 5 mins. This mixture was stirred at 25° C. for 18 hours. This mixture was added dropwise over ˜5 mins. to a mixture of 40 g of MeOH containing morpholine (9 g, 103 mmol, 1.5 moles per acrylate) cooled at 4° C. This resulting mixture was stirred at RT for 18 hours. This mixture was evacuated of volatiles with a rotary evaporator followed by high vacuum for 3 hours to give 24 g of crude material. An aliquot of this mixture (900 mg) was purified using a Sephadex™ LH-20 column (void volume 105 mL) in MeOH taking 30×3 mL fractions. A TLC (MeOH) indicated that the desired product was present in fractions 1-16. These collected fractions were evacuated of volatiles to a constant weight to give 180 mg (84% yield) based on (4:1), tetra-dendron adduct (Structure (12) in Scheme 3). This product gave a positive ninhydrin test indicating the presence of an active core amine hydrogen. Its spectra are as follows:

¹H NMR (300 MHz, CDCl₃): δ 2.31 (bs, 48H), 2.46 (bm, 28H), 2.60 (bm, 22H), 2.72 (bs, 10H), 3.39 (bs, 6H), 33.53 (bs, 6H), 3.62 (bs, 48H), 4.10, bm, 32H); and

¹³C NMR (75 MHz, CDCl₃): δ 32.20, 42.62, 43.20, 43.99, 49.62, 53.58, 54.07, 54.24, 62.08, 62.58, 63.07, 67.00, 171.89, 172.23.

### Example 23

### Preparation of [TREN]; (G=1); Dendri{CH₂—CH₂—CO₂—CH₂C—(CH₂CO₂CH═CH₂)₃}₄ (2.5 Moles Per NH) and Conversion to Morpholine Terminated Adduct

To a 50 ml round-bottom flask with a stir bar was added PTA [2] (7.44 g, 0.21 mmol, 2.5 moles per NH) and 15 mL of MeOH, cooled at ˜4° C., was added TREN [A] (200 mg, 1.36 mmol) in 10 mL of MeOH over about 5 mins. This resulting mixture was stirred at 25° C. for 18 hours. This mixture was added dropwise over about 5 mins. to a mixture of 40 g of MeOH containing morpholine (11 g, 126 mmol, 1.5 moles per acrylate), cooled at 4° C. This mixture was stirred at 25° C. for 18 hours. The volatiles of this mixture were removed using a rotary evaporator and high vacuum for 4 hours to give 14.6 g of crude material. An aliquot of this material was purified using a Sephadex™ LH-20 column (void volume 105 mL) in MeOH taking 30×3 mL fractions. A TLC (MeOH) indicated that the desired product eluded in fractions 1-16. These collected fractions were evacuated to a constant weight to give 220 mg (95% yield) based on a (5:1) penta-dendron adduct (Structure (13) in Scheme 3). This product gave a positive ninhydrin test indicating the presence of an active core amine hydrogen. Its spectra are as follows:

¹H NMR (300 MHz, CDCl₃) δ 2.41 (bs, 60H), 2.49 (bs, 55H), 2.61 (bm, 28H), 2.73 (bs, 15H), 3.40 (bm, 8H), 3.52 (bm, 10H), 3.62 (bs, 60H), 4.10 (bm, 41H); and

¹³C NMR (75 MHz, CDCl₃) δ 32.20, 42.62, 43.21, 43.96, 49.65, 51.87, 53.59, 54.07, 54.42, 62.09, 62.58, 63.07, 66.91, 67.01, 171.90, 171.98, 172.24.

### Example 24

### Synthesis of G=1.0 [PETGE [C] Core, Epoxy Surface] Dendrimer from PETGE [C] and PETGE-PIPZ [14]

To a 50 mL microwave quartz reactor was added PETGE [C] (2.88 g, 8 mmol) and MeOH (15 mL). The reactor was equipped with a stir bar. After stirring for 5 mins. at RT, a solution of PETGE-PIPZ [14] (0.704 g, 1 mmol) in MeOH (5 mL) was added dropwise over 10 mins. in five portions, stirred for 5 mins. and then placed in a microwave. The reaction mixture was irradiated under, 150 W, 8 mins., 45° C. Progress of the reaction was monitored by MALDI-TOF and it showed 2, 3 and 4-arm products. The reaction mixture was again irradiated under identical conditions and analysis showed the same results. TLC (1:1 of NH₄OH and MeOH with ninhydrin spray, Rf=0.76 for G=1.0 epoxy (olive green in pink) and Rf=0.4 for PETGE-PIPZ (light pink) indicated complete consumption of the PETGE-PIPZ. Solvent was removed on a rotary evaporator under reduced pressure at low temperature (45° C.). The resulting reaction mixture was purified through Sephadex (LH-20) in MeOH. After eluting 360 mL (v/v=360 mL), fractions were collected in 5 mL quantity. The first 28 fractions were mixed and solvent was removed on a rotary evaporator under reduced pressure at low temperature (45° C.) to give pale yellow color viscous liquid, which was further dried under high vacuum gave 1.32 g (61.5%) of pale yellow color foamy solid [15]. This sample was stored in DCM at 4° C. Its spectra are as follows:

¹H NMR (300 MHz, CDCl₃): δ 2.23 (H, m), 2.40 (H, m), 2.55 (H, q, J=2.70H), 2.74 (H, q, 4.20 Hz), 3.07 (H, m), 3.43 (H, m), 3.64 (H, d, 2.70 Hz), 3.68 (H, d, 2.70 Hz), 3.80 (H, m); and

¹³C NMR (75 MHz, CDCl₃): δ 44.15, 44.35, 45.80, 45.87, 51.07, 51.25, 53.58, 60.87, 66.37, 70.23, 70.74, 72.24, 74.28, 74.43, 77.53; and

MALDI-TOF: C₁₀₁ H₁₈₀N₈O₄₀; Calc. 2146.5; found 2147.1170 [M]⁺, 2183.1180 ([M+Na]⁺ and 1786.0140 [one loop].

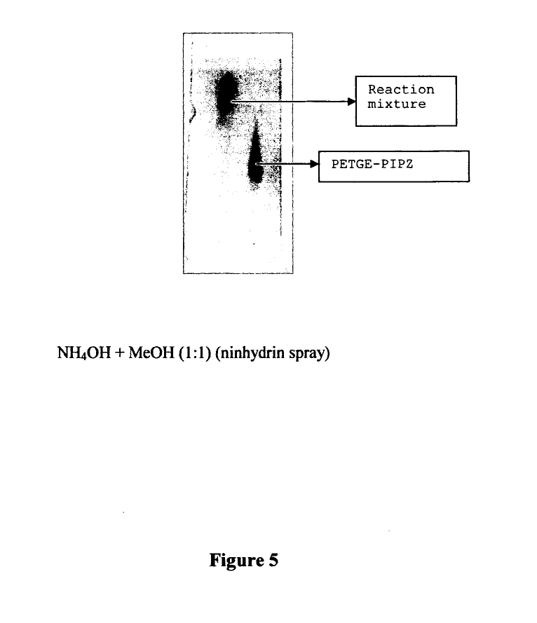

PAGE: See FIG. 5.

The following Scheme 5 illustrates this reaction.

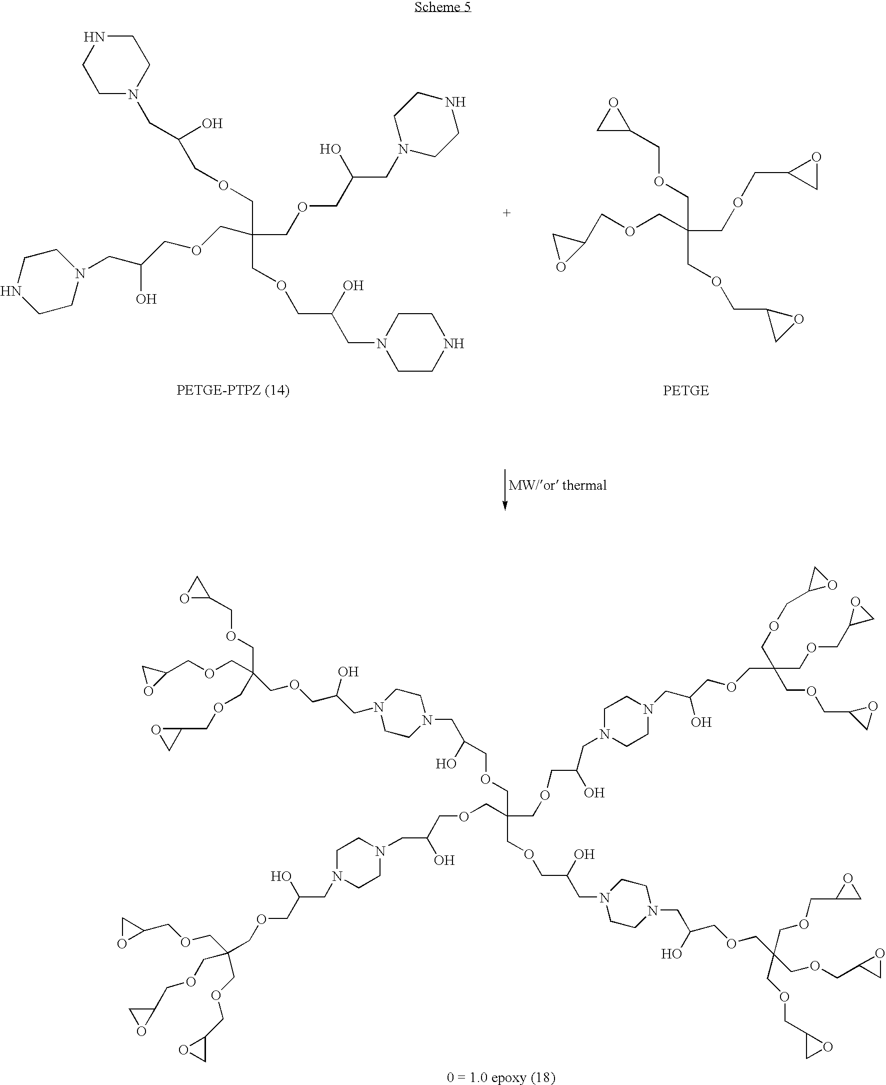

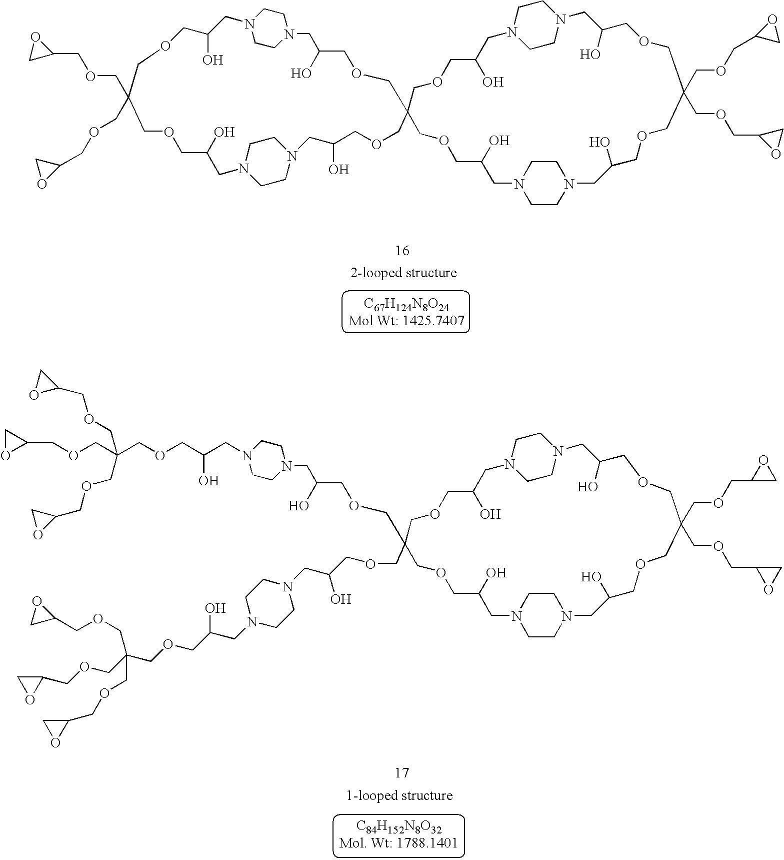

### Example 25

### Convergent Synthesis of G 1.0 Poly(Etherhydroxylamine) Dendrimer from Pentaerythritol (Tetraglycidyl Ether) and G 0 Dendrimer in DMSO/t-Butanol

Dendrimer (G=0.0) (2.112 g, 3 mmol, 12 —NH mmol) and DMSO (7 mL) were taken in a 50 mL round-bottom flask and added t-butyl alcohol (1 g). PETGE [C] (0.180 g, 0.5 mmol, 2 epoxy mmol) in DMSO (2 mL) was added into the above reaction mixture over a period of 90 mins. at RT. After completing the addition of PETGE [C], the reaction mixture was allowed to stir at RT for one hour and then heated at 45° C. for overnight. Progress of the reaction was monitored by MALDI-TOF (three drops of reaction mixture was withdrawn from reaction mixture and diluted with 1 mL of MeOH). Analysis by MALDI-TOF showed that dendrimer G=1.0 is formed, but other possible low molecular weight products (PETGE-(G=0)₂₋₃) were also noticed. Heating continued for 3 days. The reaction mixture was diluted to 5% solution by adding DI water and subjected to UF (3K). Separation of excess of G=0.0 was confirmed by TLC (50% NH₄OH in MeOH). After collecting 2 L of permeate, retentate was withdrawn from UF and concentrated on a rotary evaporator to give (0.811 g) colorless sticky material. Water also removed from permeate and both the samples were analyzed by MALDI-TOF and it was found that retentate is having PETGE (G=0)₂₋₃, G=1.0 and few other small intense peaks at high molecular compounds. Also from the permeate it was found that it has G=0 and PETGE (G 0)₂₋₃ and high molecular compounds. Its spectra are as follows:

¹H NMR (300 MHz, D₂O): δ 2.32 (H, t, J=5.10 Hz), 2.40 (H, m), 2.69 (H, m), 3.36 (H, m), 3.73 (H, m), 3.89 (H, m); and

¹³C NMR (75 MHz, D₂O): δ 43.56, 43.96, 45.29, 45.46, 52.41, 53.31, 60.58, 61.20, 62.77, 66.94, 67.16, 70.01, 70.45, 72.52, 73.94, 74.39; and

IR (Neat): λₘₐₓ 3375, 2939, 2883, 2822, 1649, 1562, 1536, 1460, 1424, 1321, 1296, 1111, 1014, 860, 819, 799 cm⁻¹; and

MALDI-TOF: 1793.6070 (two loop+Na) [19], 2498.7140 (one loop+Na) [18], 3203.0200 (G 1+Na) amu.

The following Scheme 6 illustrates this reaction.

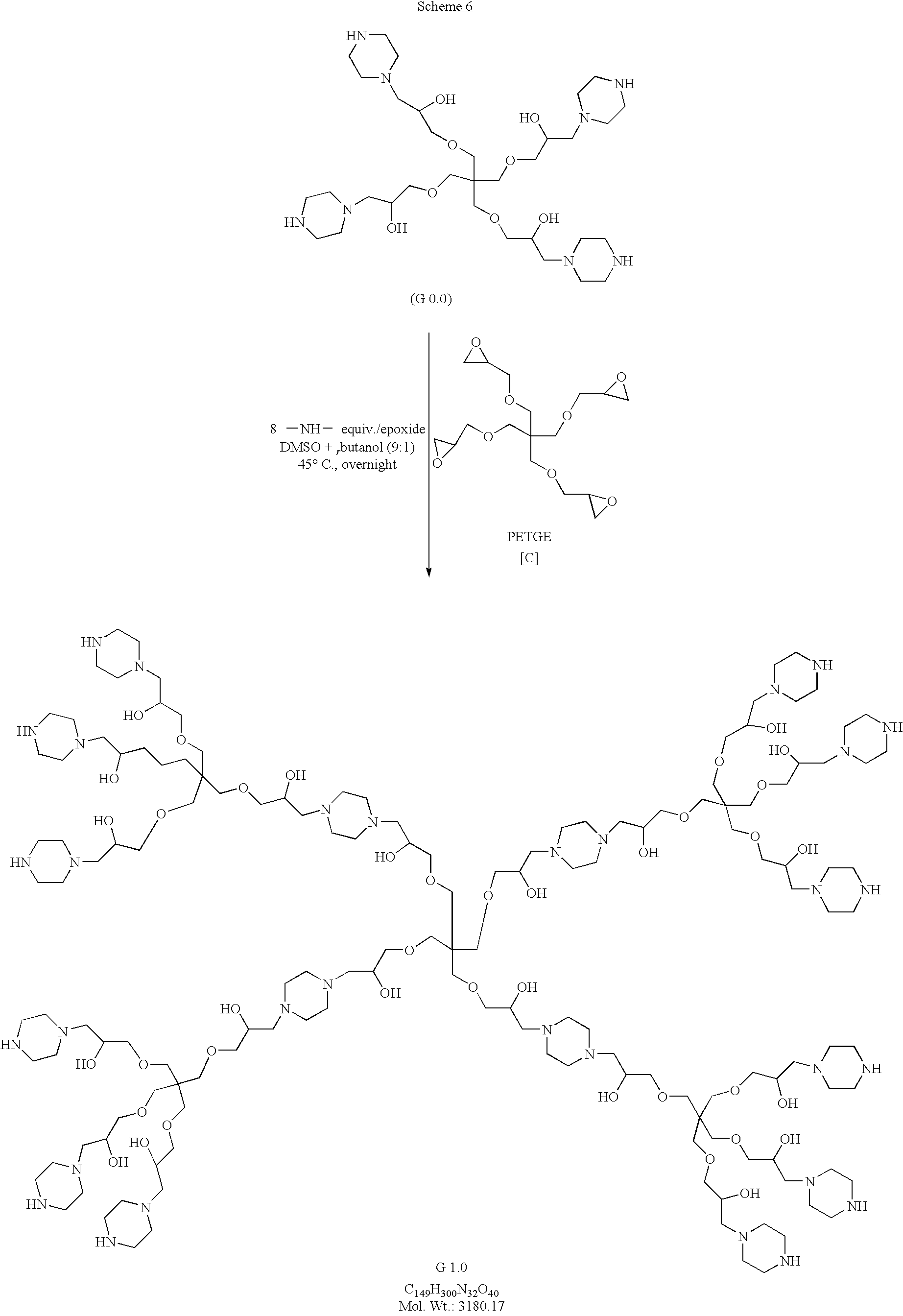

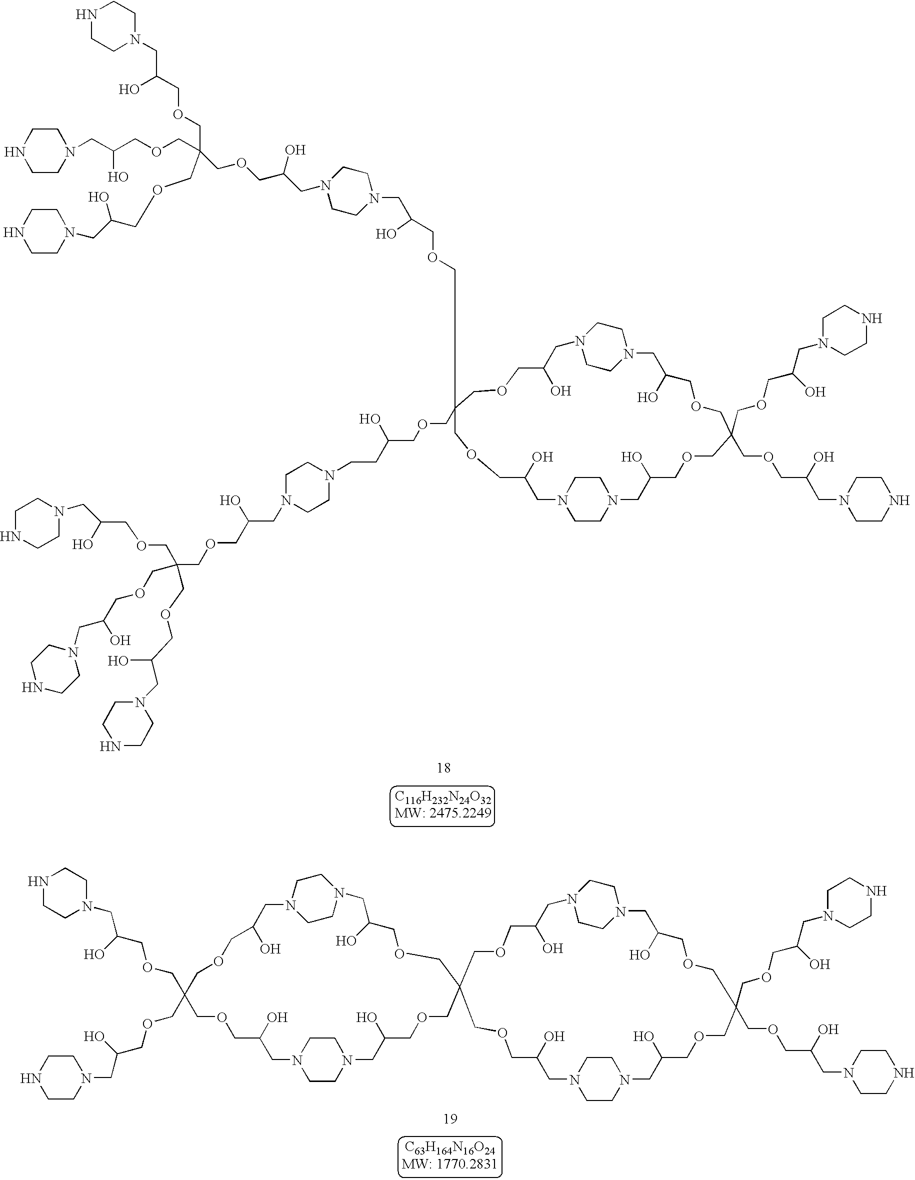

### Example 26

### Synthesis of TMPTGE [D] Core Amine Surface (G=2.0) Dendrimer with TREN [A]

A 100 mL single neck round-bottom flask was charged with TREN [A] (17.05 g, 116.82 mmol, 60 —NH₂ equiv./ester) and MeOH (40 mL, Fisher) and a stir bar. After adding MeOH an exothermic reaction resulted. After 20 mins., a solution of G=1.0 ester [20] (0.846 g, 0.97 mmol, 5.84 ester mmol) in MeOH (10 mL, Fisher) was added dropwise over a period of 1 hour at RT. After completing the addition, the reaction mixture was placed in an oil-bath and heated at 50° C. for 3 days. Progress of the reaction was monitored by IR. IR spectra on crude sample indicated absence of ester band (1740 cm⁻¹) and presence of amide band (1567 cm⁻¹). MALDI-TOF analysis indicated mass for G=2.0 and looped compound at 1348 [M+Na]⁺, 1201 [M+Na]⁺, (one and two looped). and a weak intense peak for crosslinked products. The reaction mixture was diluted with MeOH (700 mL) and subjected to UF (1K). After collecting 1.8 L of permeate, retentate was withdrawn from UF, and the solvent was removed on a rotary evaporator to give a pale yellow color, thick liquid, which was further dried under high vacuum to give dendrimer [21], 1.41 g (98.94%). Removal of solvent from permeate gave 16.1 g of TREN [A]. The spectra of these products are as follows:

### Dendrimer [21]:

¹H NMR (300 MHz, CD₃OD): δ 0.86 (3H, bt), 1.38 (2H, bs), 2.32-2.60 (H, m), 2.67-2.76 (H, m), 3.29-3.34 (H, m), 3.82 (3H, bs); and

¹³C NMR (125 MHz, CD₃OD): δ 8.14, 24.06, 38.57, 38.63, 39.98, 40.16, 44.59, 54.00, 55.09, 55.28, 57.21, 58.02, 60.19, 63.05, 63.28, 69.38, 69.94, 72.52, 72.96, 75.00, 173.76, 173.86, 174.03; and

IR (Neat): νₘₐₓ 3298, 2934, 2842, 1659, 1572, 1536, 1470, 1388, 1357, 1311, 1116, 973, 819 cm⁻¹; and

MALDI-TOF: C₆₃H₁₄₃N₂₇O₁₂ Calc. 1470.9843.; found 1494.2270 [M+Na]⁺, 1348.022 [M+Na]⁺(one looped) [22], 1201.0970 [M+Na]⁺(two looped) amu [23]; and

The following Scheme 7 illustrates this reaction.

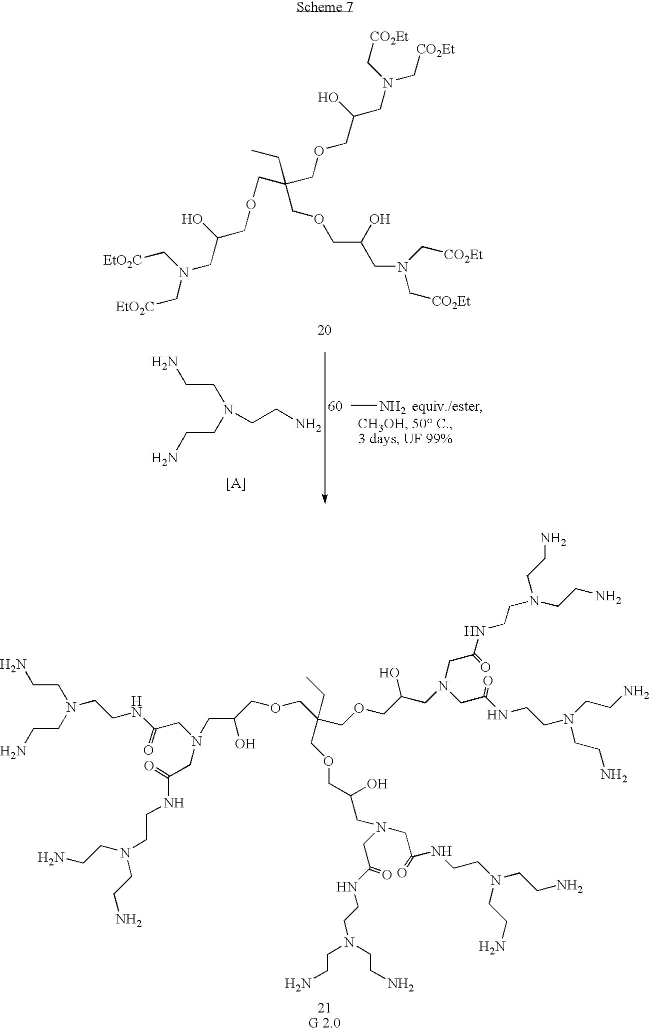

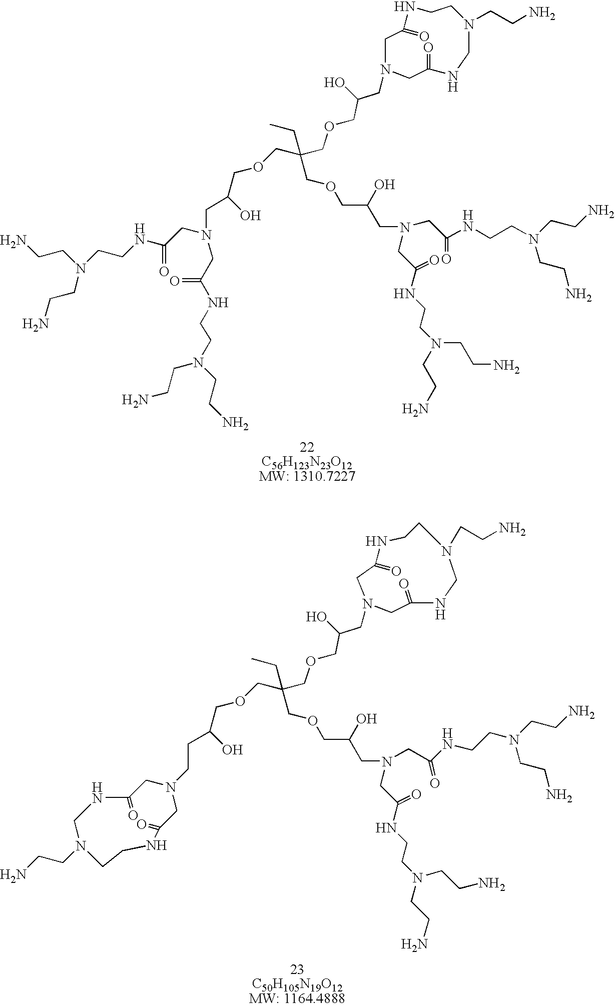

### Example 27

### Synthesis of PETGE [C] Core Amine Surface (G=2.0) Dendrimer with TREN [A]

A 250 mL single neck round-bottom flask was charged with TREN [A] (52.26 g, 358 mmol, 120-NH₂ equiv./ester) and MeOH (50 mL, Fisher) and a stir bar. After adding MeOH an exothermic reaction resulted. After 30 mins., a solution of G=1.0 ester [25] (1.25 g, 1.12 mmol, 8.95 ester mmol) in MeOH (10 mL, Fisher) was added dropwise over a period of 1 hour at RT. After stirring it overnight at RT, MALDI-TOF analysis showed mass for perfect structure (G=2.0), one looped and two looped products, and no crosslinked products were found. At this time IR spectrum also was recorded and it showed the presence of amide band (1575 cm⁻¹) and absence of an ester band (1740 cm⁻¹). Stirring continued for 36 hours. The reaction mixture was diluted to 5% w/w solution in MeOH and subjected to UF (1K). After collecting 3.5 L of permeate, retentate was withdrawn from UF, solvent was removed on a rotary evaporator, dried under high vacuum to give pale yellow color dendrimer [26] as a foamy solid, 2.02 g (94%). Its spectra are as follows:

### Dendrimer [26]:

¹H NMR (500 MHz, CD₃OD): δ 2.49-2.59 (H, m), 2.62 (H, bt), 2.66 (H, s), 2.68 (H, s), 2.69 (H, s), 2.70 (H, s), 2.73-2.82 (H, m), 3.29-3.47 (H, m), 3.82 (H, bs); and

¹³C NMR (125 MHz, CD₃OD): δ 38.64, 40.19, 48.48, 49.85, 53.94, 55.10, 55.29, 57.66, 58.10, 60.23, 63.06, 69.33, 71.41, 75.11, 173.70, 173.80, 173.97; and

IR (Neat): νₘₐₓ 3313, 3078, 2934, 2868, 1649, 1557, 1541, 1475, 1449, 1362, 1306, 1163, 1101, 978, 818 cm⁻¹, and

MALDI-TOF: C₈₁H₁₈₄N₃₆O₁₆ Calc. 1918.5607; found 1941.8310 [M+Na]⁺, 1794.7280 [M+Na]⁺(one looped) [27], 1648.5250 [M+Na]⁺(two looped) amu [24]; and

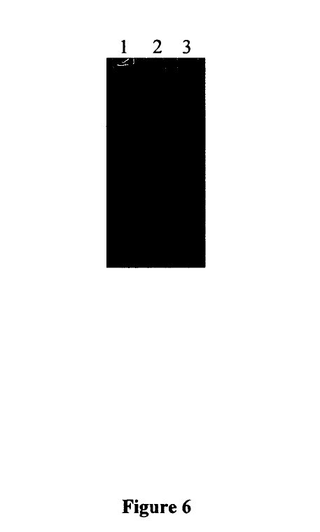

PAGE: See FIG. 6, lane-3.

The following Scheme 8 illustrates this reaction.

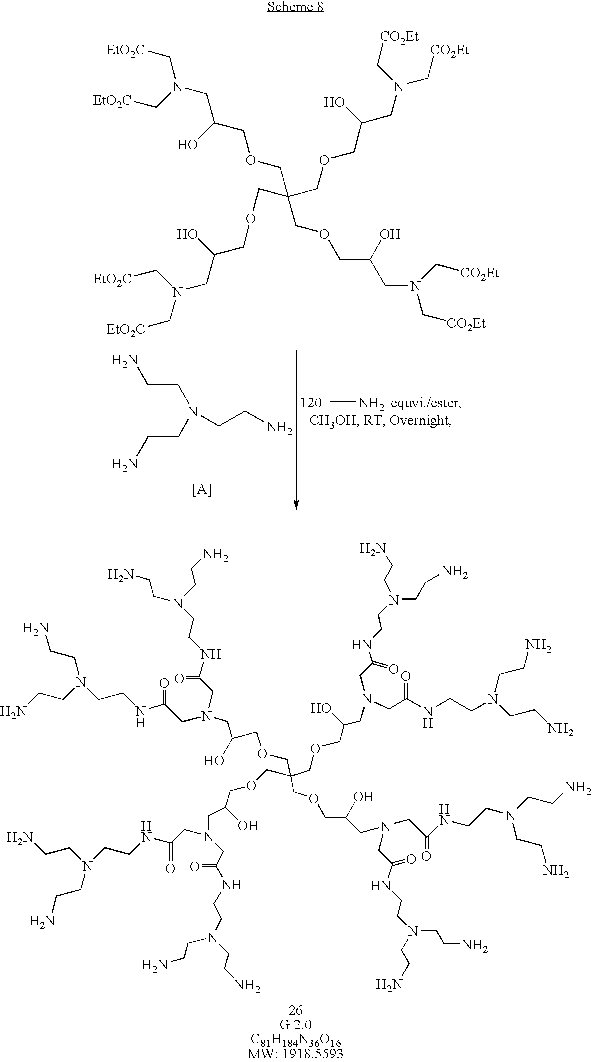

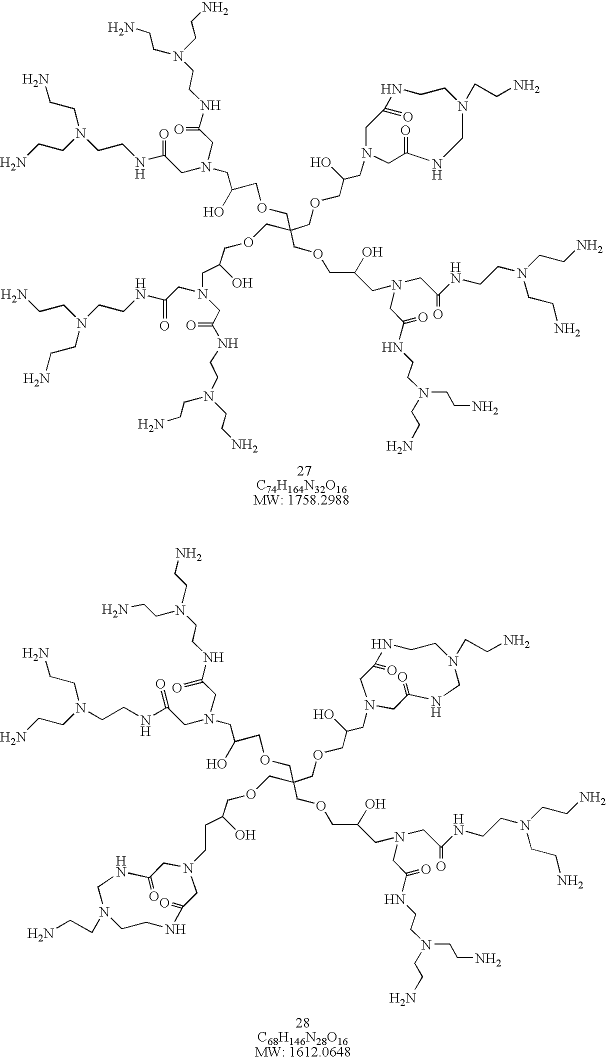

### Example 28

### Synthesis of TMPTGE [D] Core Amine Surface (G=1.0) Dendrimer with TREN [A]

To a 25 mL round-bottom flask with a stir bar was added TMPTGE [D] (1.81 g, 6.0 mmol) dissolved in 6 mL of MeOH. To this stirred mixture cooled to 40° C. was added dropwise over 10 min a solution of TREN [A] (964 mg, 6.6 mmol) in 20 mL of MeOH. This mixture was allowed to warm to RT and stirred overnight under N₂ gas. Volatiles were removed under high vacuum to give a viscous oil. The oil was dissolved in MeOH and purified on a Sephadex™ LH-20 column using MeOH as mobile phase. The eluent was collected in 8-mL fractions. Fractions 1-12 were combined and stripped on a rotary evaporator, followed by high vacuum to give the dendrimer cryptand in a yield of 1.3 g (49% yield). Its spectra are as follows:

¹H NMR (300 MHz, CD₃OD) δ 0.86 (bs, 3H), 1.41 (bm, 2H), 2.3-3.0 (bm, 14H), 3.1-3.6 (bm, 12H), 3.7-3.8 (bm, 4H).

¹³C NMR (75 MHz, CD₃OD) δ 6.95, 22.68, 43.18, 52.57, 53.82, 55.48, 68.84, 71.84, 74.48.

MALDI-TOF MS: C₂₁H₄₄N₄O₆Calc. 448.60. found: 450.81 (M+H).

The following Scheme 9 illustrates this reaction.

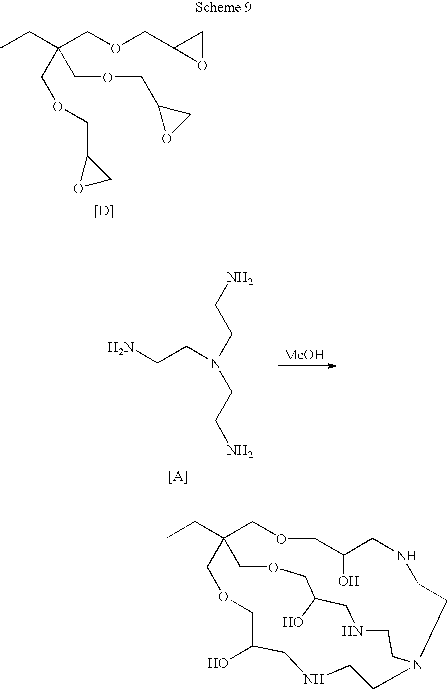

### Example 29

### Synthesis of TMPTGE [D] Core Amine Surface (G=1.0) Dendrimer with DETA [B]

To a 50 mL round-bottom flask containing a stir bar was added DETA [13] (6.2 g, 60 mmol, 3 equiv. per epoxide) and 6 g of MeOH. This mixture was cooled to 4° C. TMPTGE [D] (2.0 g, 6.62 mmol, 19.86 mmol epoxide) dissolved in 2 g of MeOH was added dropwise over 3 min, the mixture stirred at 4° C. for 1 hour, and another 20 hours at 40° C. under N₂ gas. The reaction mixture was cooled to RT and volatiles were removed by rotary evaporation, followed by Kugelrohr distillation at 210° C. for 1 hour to remove unreacted DETA [B]. The residue, consisting of the 3:1 adduct (minor product) and the 2:1 cryptand adduct (main product), gave a total weight of 3.36 g. Its spectra are as follows:

¹H NMR (300 MHz, CD₃OD) δ 0.87 (bm, 3H), 1.40 (bm, 2H), 2.4-2.8 (bm, 16H), 3.28-3.62 (bm, 12H), 3.8 (bm, 3H).

¹³C NMR (75 MHz, CD₃OD) δ 6.76, 6.91, 22.350, 22.79, 38.96, 40.70, 43.41, 43.53, 48.45, 48.49, 48.65, 48.68, 51.61, 52.50, 52.61, 57.32, 62.64, 68.91, 71.60, 74.25, 74.34.

MALDI-TOF MS: C₂₃H₅₂N₆O₆, 508.39; Calc. 508.70, found 509 (M+1), 531 (M+Na), 612 (M+1; 3:1 adduct) and 634 (M+Na) amu.

The following Scheme 10 illustrates this reaction.

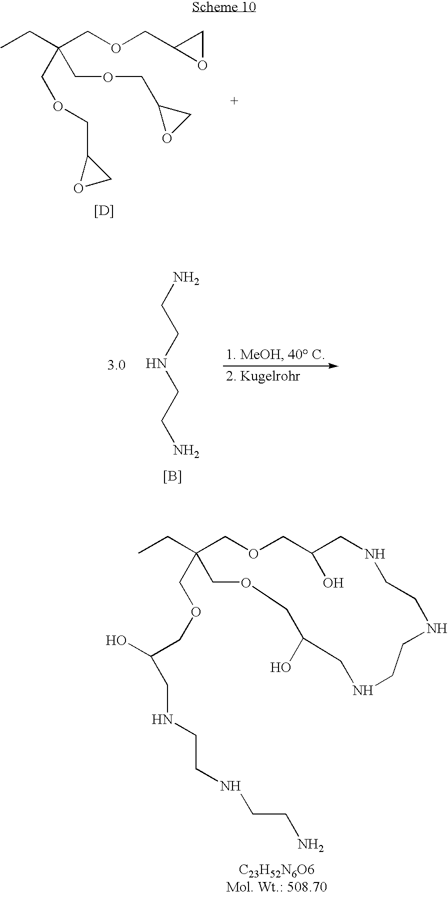

Although the invention has been described with reference to its preferred embodiments, those of ordinary skill in the art may, upon reading and understanding this disclosure, appreciate changes and modifications which may be made which do not depart from the scope and spirit of the invention as described above or claimed hereafter.

1. Dendrimers and dendrons comprising a looped, macrocyclic structure in one or more branches of the formula 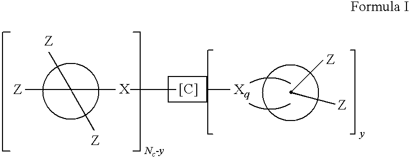
wherein:
[C] is the core of the dendrimer or dendron that may have more than 1 branch arm depending on the number of core-XR functionalities present;
XR represents the core functionalities and when R═H is a mono-alkyl amine, an α,ω-alkylenediamine, a poly(thiol) moiety, a mono- or poly-alkylene-X moiety, or when R═C is a strained ring having at least 1× moiety as part of the ring;
X is N, S or O;
N_{c} is the multiplicity of the core and is from 1 to 1,000;
y is the number of macrocyclic loops and is from 1 to Z/2;
q is from 1-3;
Z is the surface of the dendrimer or dendron and is a poly(ester)- or poly(ether)-substituted terminal functionality, which may be from 1 to the theoretical number possible.

2. The dendrimers or dendrons of claim 1 wherein Z is poly(ester)- or poly(ether)-(acrylate, amine, piperazine, epoxy, aziridine, thioether or morpholine) moiety or any functionality derived from nucleophilic addition or reaction with an epoxy, acrylate, thioether, or aziridine moiety.

3. The dendrimers or dendrons of claim 1 wherein y is 1 or 2.

4. The dendrimers or dendrons of claim 1 wherein X is N.

5. The dendrimers or dendrons of claim 1 wherein the core is a mono-alkyl amine, an α,ω-alkylenediamine, a polyalkylene amine, a poly(thiol), or a poly(epoxy) core.

6. A process for making the dendrimers and dendrons of claim 1 wherein the compounds of Formula I are prepared by reacting a dendrimer or dendron core having one or more core-XR functionalities as defined in claim 1 available for reaction with a branch cell reagent, where the molar ratio of branch cell reagent to core-XR is from about 0.5:1 to about 2:1, in the presence of a suitable solvent.

7. The process of claim 6 wherein the core is EDA, HDA, DETA, TREN, PETGE, TMPTGE, PEHAM, PAMAM or PEI.

8. The process of claim 1 or 7 wherein the surface group terminal functionality is a poly(ester)- or poly(ether)-(acrylate, amine, piperazine, epoxy, aziridine, thioether, or morpholine) moiety.

9. The process of claim 6 wherein the branch cell reagent is PTA, PETGE, TMPTA, TMPTGE, PPT, TREN or DETA.

10. The process of claim 6 wherein the solvent is methanol.

11. The process of claim 6 wherein the core is a mono-alkyl amine core and the branch cell reagent is PTA, PETGE, TMPTA, PPT or TMPTGE and the molar ratio of branch cell reagent to core-XR functionality is from about 0.5:1 to about 1:1.

12. The process of claim 6 wherein the core is an α,ω-alkylenediamine core, the branch cell reagent is PTA, PETGE, TMPTA, PPT or TMPTGE and the molar ratio of branch cell reagent to core-XR functionality is about 2:1.

13. A method of using dendrimers or dendrons as claimed in any one of claims 1-5 as sensors or detection research reagents or f as in vivo, in vitro or ex vivo diagnostic materials.

14. A method of using dendrimers or dendrons as claimed in any one of claims 1-5 as transfection agents in research or as therapeutic or diagnostic agents.

15. A method of using dendrimers or dendrons as claimed in any one of claims 1-5 as additives in cosmetic, ink, toner, plastic, paper, or coating applications.

16. A method of using dendrimers or dendrons as claimed in any one of claims 1-5 as a filtration material in water remediation applications.

17. A method of using dendrimers or dendrons as claimed in any one of claims 1-5 as agents in therapeutic applications.

18. A method of using dendrimers or dendrons as claimed in any one of claims 1-5 as chelating agents.

19. The dendrimers or dendrons of claim 18 where the chelate or complex is formed at any location Core, X, or Z on the dendrimer or dendron with: (a) metals or their ions, (b) nucleic acids, (c) dyes or pigments, (d) pharmaceuticals, (e) cosmetic ingredients or (f) combinations of these chelates with the dendrimer or dendron.
